(19) United States
(12) Patent Application Publication
(10) Pub. No.: US 2019/0002834 A1
(43) Pub. Date: Jan. 3, 2019

(54) METHOD FOR PREPARING POPULATION OF STEM CELL SPHEROIDS

(71) Applicants: KURARAY CO., LTD., Okayama (JP); I Peace, Inc., Palo Alto, CA (US)

(72) Inventors: Koji TANABE, Palo Alto, CA (US); Kenta SUTO, Palo Alto, CA (US)

(21) Appl. No.: 16/067,103

(22) PCT Filed: Dec. 28, 2016
(86) PCT No.: PCT/JP2016/089185

§ 371 Date: Jun. 28, 2018

Related U.S. Application Data
(60) Provisional application No. 62/272,524, filed on Dec. 29, 2015.

(51) Int. Cl. C12N 5/074

(52) U.S. Cl. CPC C12N 5/0696; C12N 2509/00; C12N 2506/45; C12N 2535/00; C12N 2501/115

(57) ABSTRACT

The present invention relates to a method for forming cell aggregates. In the method, two or more clusters of cells are distributed into each of two or more compartments. The two or more clusters of cells are brought close to each other in each of the compartments. The two or more clusters of cells brought close to each other are clumped or assembled. The clumped or assembled clusters of cells are allowed to grow to thereby form cell aggregates. The clusters of cells to be distributed are separated from each other and mixed with each other. The clusters of cells each include a stem cell.

### TECHNICAL FIELD

The present invention relates to a method for preparing a population of cell aggregates of stem cells.

### BACKGROUND ART

A method for forming an embryoid body by clumping or assembling pluripotent stem cells is known (Patent Literature 1). In this method, an enzyme is used for the embryoid body (EB) so that cells are isolated into single cells (claim 9). The individualized cells are clumped or assembled again (claim 18). This method is suitable for differentiating pluripotent stem cells into endothelial cells.

### CITATION LIST

### Patent Literature

Patent Literature 1: Published Japanese Translation of PCT International Publication for Patent Application, No. 2012-519005

### Non Patent Literature

Non-Patent Literature 1: Kenji Osafune, Leslie Caron, Malgorzata Borowiak, Rita J Martinez, Claire S Fitz-Gerald, Yasunori Sato, Chad A Cowan, Kenneth R Chien & Douglas A Melton, “Marked differences in differentiation propensity among human embryonic stem cell lines”, Nature Biotechnology, Published online: 17 Feb. 2008, 26, 313-315

### SUMMARY OF INVENTION

### Technical Problem

In the method described above, in order to prepare a large number of embryoid bodies, a plurality of clusters of cells are formed by dissociating an embryoid body and the clusters of cells are allowed to grow into new embryoid bodies. However, in general, the embryoid bodies include cells that have already started to differentiate. Therefore, the method is not suitable as a method for increasing the number of cell aggregates of pluripotent stem cells, while substantially maintaining the undifferentiated state of the cell aggregates of pluripotent stem cells.

The present inventors have considered as follows in the process of achieving the present invention. Non-Patent Literature 1 discloses that the degree of advancement of differentiation varies depending on the culture period of cells (Supplementary FIG. 1 in Non-Patent Literature 1). If the cells having different degrees of advancement of differentiation are passed through, the degree of advancement of differentiation is taken over by cell aggregates formed after the passage. Therefore, it is estimated that the uniformity in the undifferentiated state in the cell aggregates is lowered every time the passage is repeated. This seems to be because the direction of differentiation or undifferentiation varies depending on the size of cell aggregates. It is considered that the reason why this phenomenon occurs is that since nutrition necessary for cells to survive is not appropriately diffused to the inside of each cell aggregate, the nutrition is not supplied to the center of each cell aggregate. Further, the fact that gases and unnecessary substances are also not diffused is an obstacle to the survival of cells located inside the cell aggregate. In addition, if the cell aggregates grow excessively, there is a possibility that not only differentiation but cell death occurs. On the other hand, if the cell aggregates remain small, the efficiency of the expanding culture deteriorates. Accordingly, the present inventors considered that it is important to keep the cell aggregates at a uniform size and match the timings for passage of each cell aggregate in the preparation of undifferentiated cell aggregates.

In view of the above-mentioned circumstances, an object of the present invention is to increase the uniformity in an undifferentiated state among cell aggregates during preparation of a population of cell aggregates of stem cells.

### Solution to Problem

[1] A method for preparing a population of cell aggregates of stem cells includes: distributing two or more clusters of cells into each of two or more compartments having a uniform size;

bringing the two or more clusters of cells close to each other in each of the compartments; and
allowing the two or more clusters of cells brought close to each other to be clumped or assembled and grow to form a cell aggregate. The clusters of cells to be distributed are separated from and mixed with each other, and each of the clusters of cells is formed of stem cells.

[2] The method for preparing a population of cell aggregates of stem cells according to the above item [1] further includes:

generating clusters of cells by breaking up the formed cell aggregates;

mixing the clusters of cells generated from the different cell aggregates;

distributing two or more mixed clusters of cells into each of two or more compartments;

bringing the two or more mixed clusters of cells close to each other in each of the compartments; and

clumping or assembling the two or more clusters of cells again, the two or more clusters of cells having been brought close to each other.

[3] In the method for preparing a population of cell aggregates of stem cells according to the above item [2], the cell aggregates are broken up when each of the cell aggregates has a diameter equal to or smaller than 1 mm.

[4] In the method for preparing a population of cell aggregates of stem cells according to the above item [2], during the growth of the cell aggregates, the cell aggregates are allowed to grow for a period of from 2 to 14 days.

[5] In the method for preparing a population of cell aggregates of stem cells according to the above item [2], wherein during the growth of the cell aggregates, the cell aggregates are allowed to grow for a period of from 3 to 7 days.

[6] In the method for preparing a population of cell aggregates of stem cells according to the above item [2], a process of breaking up the cell aggregates, mixing the clusters of cells, bringing the clusters of cells close to each other, and clumping or assembling the clusters of cells again is repeated once or twice or more.

[7] In the method for preparing a population of cell aggregates of stem cells according to the above item [1], the stem cells are cultured in a plate to form a colony;

the clusters of cells are generated by dissociating the colony;

the generated clusters of cells are mixed; and

the clusters of cells are used for the distribution.

[8] In the method for preparing a population of cell aggregates of stem cells according to the above item [7], the colony is broken up by physical dissociation, and an enzyme treatment is not applied onto the colony.

[9] In the method for preparing a population of cell aggregates of stem cells according to the above item [7], the colony is broken up only by an enzyme treatment, and physical dissociation is not applied onto the colony.

[10] In the method for preparing a population of cell aggregates of stem cells according to the above item [7], when the colony is broken up, an enzyme treatment and physical dissociation are applied onto the colony.

[11] In the method for preparing a population of cell aggregates of stem cells according to the above item [1], the compartments are each formed by a hole of a plate; the hole is one of a through-hole and a recess;

the hole has a top opening formed in a top face of the plate;

the top openings of the holes of the compartments have an equal area; and

the top opening has a diameter of 1.5 mm or less.

[12] In the method for preparing a population of cell aggregates of stem cells according to the above item [1], the compartments are each formed by a through-hole of a plate;

the through-hole has a bottom opening formed in a bottom of the plate;

the bottom opening has a diameter of 1 mm or less; and

the cell aggregates are recovered from the plate by causing the cell aggregates to pass through the bottom opening.

[13] The method for preparing a population of cell aggregates of stem cells according to the above item [12], in which the clusters of cells are cultured in a culture solution dispensed in the compartment;

the culture solution forms a droplet;

the droplet adheres to the bottom opening and projects from the bottom opening so as to hang down therefrom; and

the bottom of the compartment is formed by a meniscus of the droplet.

[14] In the method for preparing a population of cell aggregates of stem cells according to the above item [1], an inscribed sphere in each of the compartments has a diameter in a range from 5×10¹ μm to 1×10³ μm, and the inscribed sphere contacts the bottom of the corresponding compartment.

[15] In the method for preparing a population of cell aggregates of stem cells according to the above item [1], the clusters of cells are cultured in a culture solution dispensed in each of the compartments;

the culture solution is joined with a culture solution dispensed in a reservoir compartment through a top portion of each of the compartments; and

no cells are present in the culture solution in the reservoir compartment.

[16] In the method for preparing a population of cell aggregates of stem cells according to the above item [1], the compartments are each formed by a hole of a plate;

the hole is one of a through-hole and a recess;

the hole has a top opening formed in a top face of the plate; and

during the distribution, the top face is covered with a suspension containing the clusters of cells.

[17] In the method for preparing a population of cell aggregates of stem cells according to the above item [16], the suspension contains one to 5000 clusters of cells per unit area (1 cm²) of the top face.

[18] The method for preparing a population of cell aggregates of stem cells according to the above item [1], in which the clusters of cells are cultured in a culture solution dispensed in the compartment; and

extracellular matrixes are suspended or dissolved in the culture solution.

[19] A cell culture method including:

forming a cell aggregate from stem cells; and

differentiating the stem cells while performing suspension culturing of adherent culturing, in which,

during the formation of the cell aggregate, two or more clusters of cells are distributed into each of two or more compartments having an equal size,

the two or more clusters of cells are brought close to each other in each of the compartments,

the two or more clusters of cells brought close to each other are clumped or assembled and allowed to grow to form a cell aggregate,

before the distribution, the clusters of cells are separated from each other and are mixed with each other, and

each of the clusters of cells includes stem cells.

[20] The cell culture method according to the above item [19], in which cells in the cell aggregates are further differentiated into one of ectoderms, mesoderms, and endoderms in the compartment.

[21] A population of cell aggregates, in which:

one cell aggregate is selected from the population;

10 or more cells are selected from cells in the selected cell aggregates;

a positive rate of the 10 or more cells is measured by determining whether or not at least one of pluripotent stem cell markers of Nanog, Oct3/4, and TRA-1-60 is positive for the cells; and

when the positive rate is measured from the population three times, an average of the three positive rates is 80% or higher.

[22] In the population of cell aggregates according to the above item [21], ten cell aggregates are selected from the population;

when it is determined as to whether or not at least one of pluripotent stem cell markers of Nanog, Oct3/4, and TRA-1-60 is positive for the ten selected cell aggregates, a positive rate of the marker is 80% or higher.

[23] In the population of cell aggregates according to the above item [21], a ratio of embryoid bodies induced from the cell aggregates by an in vitro differentiation-inducing system is 80% or higher, and the embryoid bodies are cell aggregates containing mixed tissues of three germ layers.

### Advantageous Effects of Invention

According to the present invention, it is possible to make the sizes of cell aggregates uniform in preparation of a population of cell aggregates of stem cells. Therefore, the present invention can improve the uniformity of undifferentiated states of the cell aggregates. Thus, the present invention is suitable for preparation of undifferentiated cell aggregates.

### BRIEF DESCRIPTION OF DRAWINGS

FIG. 1 is a flowchart showing a method for preparing a population of cell aggregates;

FIG. 2 is a sectional view showing a culturing device;

FIG. 3 is an enlarged sectional view showing clusters of cells and a plate;

FIG. 4 is an enlarged sectional view showing cell aggregates and a plate;

FIG. 5 is a diagram showing a state in which a chamber and a tray are separated;

FIG. 6 is a diagram showing a state in which a chamber and cell aggregates are separated;

FIG. 7 is an enlarged view showing a state in which a chamber and cell aggregates are separated;

FIG. 8 is a graph showing a distribution of sizes of cell aggregates;

FIG. 9 shows observation images 1 of cell aggregates according to an example;

FIG. 10 shows observation images 2 of cell aggregates according to an example;

FIG. 11 shows observation images 3 of cell aggregates according to an example;

FIG. 12 shows observation images 4 of cell aggregates according to an example;

FIG. 13 shows 2-parameter histograms of FACS;

FIG. 14 shows observation images of cells obtained from cell aggregates according to an example;

FIG. 15 shows observation images of populations of cell aggregates according to an example;

FIG. 16 shows observation images 5 of cell aggregates according to an example;

FIG. 17 shows observation images 6 of cell aggregates according to an example;

FIG. 18 shows 2-parameter histograms of FACS;

FIG. 19 shows 1-parameter histograms of FACS;

FIG. 20 is a graph showing expression strengths of TRA-1-60;

FIG. 21A shows fluorescence observation images of cell aggregates according to an example;

FIG. 21B shows fluorescence observation images of cell aggregates according to an example;

FIG. 21C shows fluorescence observation images of cell aggregates according to an example; and

FIG. 21D is a graph in which fluorescence observations of cell aggregates according to an example are converted into numerical values.

### DESCRIPTION OF EMBODIMENTS

### [Terms]

The term “cell aggregate” described herein refers to a ball-shaped cluster of cells (block of cells) including pluripotent stem cells. The cell aggregate may have a spherical shape. The cell aggregate may be a sphere. The cell aggregate may be a spheroid. The spheroid may also be referred to as a clump. The cell aggregate is preferably formed by suspension culturing. The cell aggregate is a cluster of cells including undifferentiated pluripotent stem cells. The cell aggregate is a cluster of cells having a capability of producing various types of cells when the cell aggregate is cultured. In particular, the cell aggregate is preferably a cluster of cells including 100 to 50,000 cells.

Assume herein that the cluster of cells (block of cells) is a block in which cells are assembled and connected to each other. In the case of using the term “cluster of cells” in the following description, the cluster of cells is treated in the following manner unless explicitly specified otherwise. The term “cluster of cells” refers to a cluster of cells that is smaller than the cell aggregate. The term “cluster of cells” refers to a cluster of cells of a random size and shape. The term of “cluster of cells” includes an aggregate formed by dividing a colony or a cell aggregate.

The term “population” described herein refers to a population of clusters of cells, or a population of cell aggregates. The term “population” includes these populations held in a certain volume of liquid. The population has a predetermined density. The predetermined density is obtained by dividing the number of clusters of cells or cell aggregates by the volume of liquid.

### [Outline]

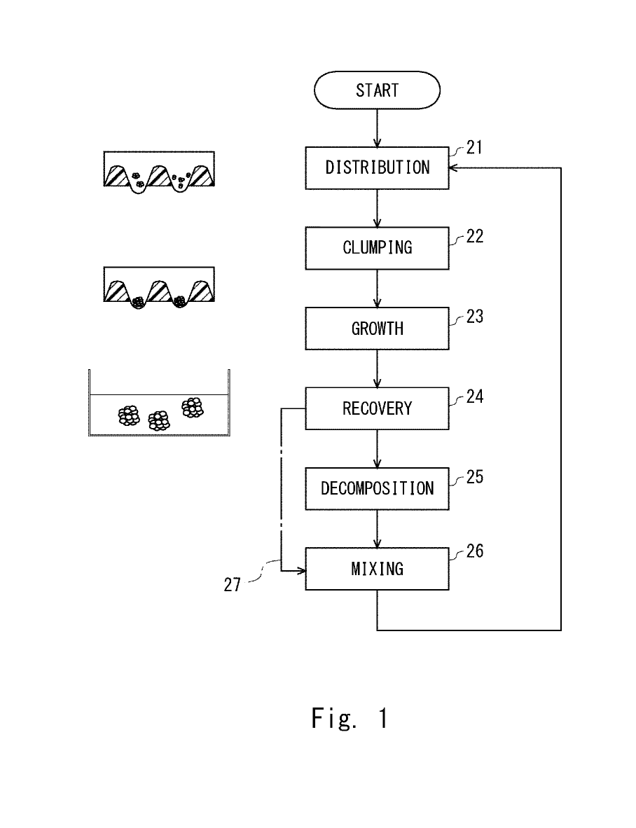

FIG. 1 is a flowchart showing a method for preparing a population of cell aggregates of pluripotent stem cells according to this embodiment. In this method, two or more clusters of cells (aggregates) are distributed into each of two or more compartments having an equal size in step 21. Thus, two or more clusters of cells are brought close to each other in each of the compartment. In step 22, the two or more clusters of cells brought close to each other are clumped or assembled. According to the method, a population of cell aggregates having an equal size is obtained, with the result that a population of cell aggregates having a homogeneity of undifferentiated state can be obtained.

After that, cell aggregates are obtained through steps 23 and 24 shown in FIG. 1. When the cell aggregates are broken up in step 25, new clusters of cells may be obtained. Further, the process may return to step 21 through step 26, and the clusters of cells may be distributed again. Thus, allowing cell proliferation in the cell aggregates and dissociation of the cell aggregates that have grown are performed as a cycle. Therefore, a large number of cell aggregates having undifferentiated states with homogeneity are obtained as a consequence of obtaining a large number of cell aggregates having an equal size.

### [Culturing Device]

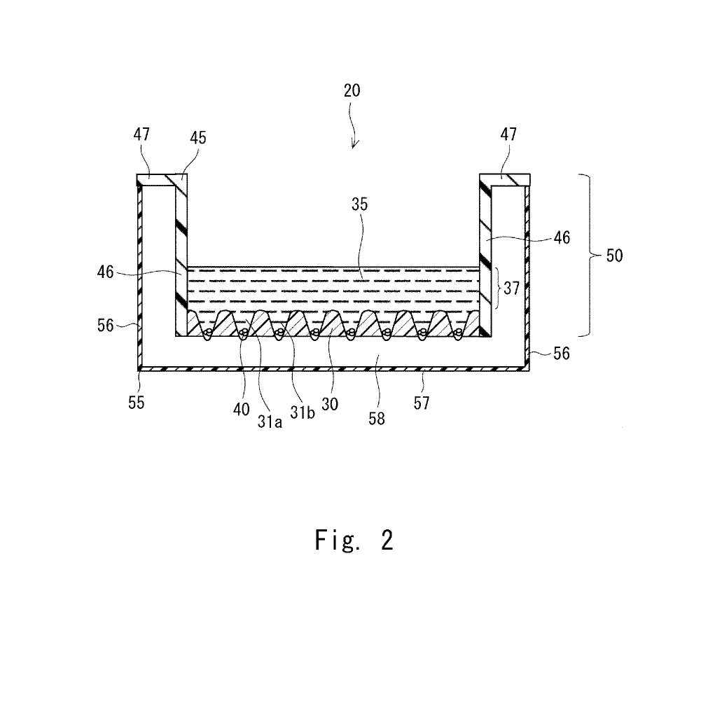

FIG. 2 shows a culturing device 20 which is suitable for carrying out the above-mentioned series of steps. The culturing device 20 includes a chamber 50 including a plate 30 and a support 45, and a tray 55. During culture of cells in the culturing device 20, the culturing device 20 may be placed in a stationary state.

The plate 30 shown in FIG. 2 includes holes as typified by holes 31a and 31b. In FIG. 2, the holes 31a and 31b are through-holes. The holes 31a and 31b may be recesses with no bottom opening. In plane view of the plate 30, the holes as typified by the holes 31a and 31b are formed in a lattice. The lattice may be a hexagonal lattice, a square lattice, or a lattice having a shape other than a hexagon and a square. In FIG. 2, the holes 31a and 31b are filled with a culture solution 35. Any culture solution may be used as the culture solution 35, as long as the culture solution is suitable for culture of pluripotent stem cells.

The support 45 shown in FIG. 2 includes side walls 46 and flanges 47. The side walls 46 surround the plate 30 and the bore of the support 45. The plate 30 is located below the bore of the support 45. The top face of the plate 30 faces the bore of the support 45. A lower portion of each of the side walls 46 is in contact with the plate 30. A lower end of each of the side walls 46 is preferably in contact with the plate 30.

The plate 30 and the support 45 which are shown in FIG. 2 integrally form the chamber 50. The plate 30 and the support 45 are preferably in contact with each other with no gap in between. The plate 30 and the support 45 integrally surround the bore of the chamber 50. The plate 30 and the support 45 may be integrally formed.

The bore of the chamber 50 shown in FIG. 2 corresponds to a reservoir compartment 37. The reservoir compartment 37 stores the culture solution 35. The top face of the plate 30 and the inner surface of each side wall 46 of the support 45 are in contact with the culture solution 35. The reservoir compartment 37 and the bore of each of the holes 31a and 31b form a continuous space.

The side walls 46 and the plate 30 shown in FIG. 2 can be integrally inserted into the bore of the tray 55. The flanges 47 are located outside of the side walls 46. The tray 55 includes side walls 56 and a bottom portion 57. The side walls 56 support the flanges 47, respectively. The flanges 47 are preferably in contact with an upper end of the corresponding side wall 56. The tray 55 supports the flanges 47. The tray 55 supports the support 45. The tray 55 supports the chamber 50. The bottom portion 57 is opposed to the plate 30. A space 58 is formed between the bottom portion 57 and the plate 30.

The plate 30 shown in FIG. 2 is preferably a resin molding. A resin to be molded is preferably any one of acrylic resin, polylactic acid, polyglycolic acid, styrene resin, acrylic styrene copolymer resin, polycarbonate resin, polyester resin, polyvinyl alcohol resin, ethylene vinyl alcohol copolymer resin, thermoplastic elastomer, vinyl chloride resin, silicone resin, and silicon resin, or a combination thereof. The plate 30 may be a molding of inorganic substance such as metal or glass. The same holds true of the other members included in the culturing device 50.

A modification treatment is preferably performed on the surface of each of the holes 31a and 31b shown in FIG. 2. The modification treatment is preferably at least one of a plasma treatment, corona discharge, and UV-ozone treatment. By the modification treatment, a functional group is formed on the surface. The functional group preferably has a hydrophilic property. The hydrophilic surface enables the clusters of cells to smoothly flow into the holes 31a and 31b. The modification treatment is preferable particularly when the opening of each of the holes 31a and 31b is small. The modification treatment is preferable particularly when resin has a hydrophobic property. The same holds true of the top face and bottom of the plate 30.

The surface of each of the holes 31a and 31b shown in FIG. 2 may be coated with a predetermined substance. The substance may be, for example, an inorganic substance, metal, a substance having a structure in which two, three, or four or more predetermined molecules are polymerized, or a combination thereof. The surface coated with the substance preferably has a certain hydrophobic property. The surface having a certain hydrophobic property facilitates formation of droplets, which are described later, even when a culture medium with small surface tension is used. The same holds true for the top face and bottom of the plate 30.

A fine structure may be formed on the surface of each of the holes 31a and 31b shown in FIG. 2. A so-called nanometer-order fine structure is preferably used. The structural unit of the fine structure is preferably in a range from 0.1 nm to 1 μm. The fine structure may be obtained by forming convex and concave shapes on the surface.

### [Compartments]

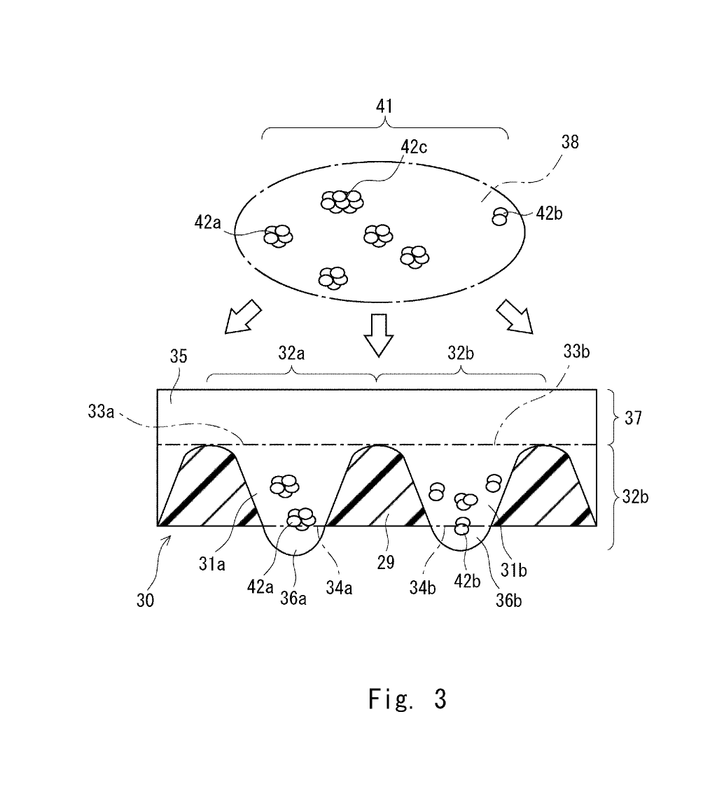

FIG. 3 is an enlarged view showing clusters of cells and the plate 30. The clusters of cells are cultured in predetermined compartments. The compartments as typified by compartments 32a and 32b are respectively formed by holes as typified by the holes 31a and 31b. The holes as typified by the holes 31a and 31b have an equal size. In this embodiment, the compartments 32a and 32b may be formed only by the holes 31a and 31b, but the structure of the compartments 32a and 32b is not limited to this.

The plate 30 shown in FIG. 3 has partition walls 29. The holes are separated by the partition walls 29. Each of the partition walls 29 is gradually narrowed toward the top portion of the plate 30 from the bottom portion thereof. Each of the holes 31a and 31b is gradually narrowed toward the bottom portion of the plate 30 from the top portion thereof

The holes 31a and 31b shown in FIG. 3 include top openings 33a and 33b, respectively, which are formed in the top face of the plate 30. The holes 31a and 31b include bottom openings 34a and 34b, respectively, which are formed in the bottom of the plate.

The top openings 33a and 33b shown in FIG. 3 preferably have an equal area. It is preferable that not only the top openings 33a and 33b, but also a plurality of top openings, preferably, all top openings of the holes have an equal area. When the top openings have an equal area, the number of cells per compartment is normalized. Accordingly, when one cell aggregate is formed in one compartment, cell aggregates having an equal size can be obtained.

The top openings 33a and 33b shown in FIG. 3 may have a circular shape. The diameter of each of the top openings 33a and 33b is preferably one of the values of 2.00 mm, 1.5 mm, 1.4 mm, 1.3 mm, 1.2 mm, 1.1 mm, 1.0 mm, 0.9 mm, 0.8 mm, 0.7 mm, 0.6 mm, 0.5 mm, 0.4 mm, 0.3 mm, 0.2 mm, and 0.1 mm or less of them.

The diameter of each of the top openings 33a and 33b shown in FIG. 3 is preferably one of the values of 10 μm, 20 μm, 30 μm, 40 μm, 50 μm, 60 μm, 70 μm, 80 μm, and 90 μm or less of them.

The top openings 33a and 33b shown in FIG. 3 may have a triangular shape, a square shape, a pentagonal shape, a hexagonal shape, or other polygonal shape, or an elliptical shape. The diameter of the inscribed circle of each of the top openings 33a and 33b can be set in a range similar to that of the diameter described above.

Even when the holes 31a and 31b shown in FIG. 3 do not have the bottom openings 34a and 34b, the top openings 33a and 33b can be adopted. Also in this case, the advantageous effect of the top openings 33a and 33b can be obtained. The top openings 33a and 33b are preferably larger than the bottom openings 34a and 34b, respectively.

### [Distribution of Clusters of Cells]

In step 21 shown in FIG. 1, a population 41 of clusters of cells shown in FIG. 3 is distributed into each of two or more compartments as typified by the compartments 32a and 32b. The population 41 includes a plurality of clusters of cells including clusters of cells 42a to 42c. The population 41 is preferably included in a suspension 38. In the suspension 38, the clusters of cells 42a to 42c are uniformly dispersed. In the population 41, a small cluster of cells 42a and a large cluster of cells 42c are mixed. The suspension 38 may include a substantially-individualized single cell(s) together with the population 41. The ratio of the number of cells in a single-cell state in the suspension 38 to the sum total of the number of cells forming clusters of cells and the number of cells in the single-cell state in the suspension 38 may be 10% or higher, 30% or higher, 50% or higher, 80% or higher, or 90% or higher.

During the distribution, the suspension 38 shown in FIG. 3 is preferably spread on the top face of the plate 30. During spreading of the suspension 38, the top face of the plate 30 is preferably coated with the suspension. The top face of the plate 30 is preferably uniformly covered with the suspension 38.

During spreading of the suspension 38, the suspension 38 preferably contains one to 5000 clusters of cells per unit area (1 cm²) of the top face. The number of clusters of cells per unit area is preferably any one of 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, and 5000 clusters.

The method of spreading the suspension 38 shown in FIG. 3 is more efficient than the method of injecting the suspension 38 into the individual compartments. The suspension 38 settles downward due to the gravity and enters into the compartments 32a and 32b. Accordingly, the suspension 38 containing the clusters of cells 42a to 42c is randomly distributed into the compartments. Further, the clusters of cells are settled in the compartments 32a and 32b. The clusters of cells settles down due to the gravity are moved away from the reservoir compartment 37 and are collected into the compartments 32a and 32b. Thus, the clusters of cells are brought close to each other.

Each of the partition walls 29 shown in FIG. 3 is preferably narrowed toward the top portion of the plate 30. Each of the partition walls 29 may have a convex sectional shape, a semicircular sectional shape, a triangular sectional shape, or the like in the vicinity of the top portion of the plate 30.

A dispersion medium forming the suspension 38 shown in FIG. 3 is filled in the compartments 32a and 32b and is dispensed in the reservoir compartment 37. The dispersion medium of the suspension 38 may be a culture solution having the same composition as that of the culture solution 35. After the distribution, a suitable culture solution may be further added to the dispersion medium in the reservoir compartment 37. After the distribution, the dispersion medium in the reservoir compartment 37 may be replaced by a suitable culture solution.

In the population 41 shown in FIG. 3, the clusters of cells to be distributed are separated from each other. These clusters of cells are mixed with each other. The population 41 includes the cluster of cells 42b that is smaller than the cluster of cells 42a. The population 41 includes the cluster of cells 42c that is larger than the cluster of cells 42a. In the population 41, the clusters of cells having different sizes are mixed.

Each of the clusters of cells 42a to 42c includes a pluripotent stem cell. The pluripotent stem cell may be an ES cell or an iPS cell. Examples of animal species of pluripotent stem cells are mammals, such as a human and a mouse, but are not limited to these. Examples of somatic cells which are the source of an iPS cell include fibroblast cells, but are not limited to these. Somatic cells may be obtained from any tissue in an individual body.

As shown in FIG. 3, the clusters of cells are cultured in the culture solution 35 which is dispensed in the compartments 32a and 32b. In FIG. 3, the clusters of cells 42a and 42b as typified by the clusters of cells are distributed into the compartments. The culture solution 35 forms droplets 36a and 36b. The droplets 36a and 36b adhere to the bottom openings 34a and 34b, respectively, and project from the bottom openings so as to hang down therefrom. The droplets 36a and 36b are projected from the bottom of the plate 30. In this embodiment, a so-called hanging-drop culture is carried out.

It may be understood that the compartments 32a and 32b shown in FIG. 3 are respectively composed of top openings 33a and 33b, bore surfaces of the holes 31a and 31b, and rounded surfaces of the droplets 36a and 36b. The surfaces face the space on the bottom side of the plate 30. The bottoms of the compartments 32a and 32b are formed by the surfaces of the droplets 36a and 36b. The droplets have the rounded surfaces because of the surface tension of the culture solution 35. That is, the surfaces of the droplets 36a and 36b form meniscuses.

As shown in FIG. 3, the culture solution 35 is filled in the compartments 32a and 32b. It may be understood that the compartments 32a and 32b are respectively composed of the culture solutions 35, which are located in the holes 31a and 31b, respectively, and the droplets 36a and 36b. In other words, the compartments 32a and 32b in which the clusters of cells 42a and 42b are cultured, respectively, are formed to be continuous with the droplets 36a and 36b outside of the plate 30.

The compartments 32a and 32b shown in FIG. 3 preferably have the following size. That is, the diameter of the inscribed sphere inscribed in each of the compartments 32a and 32b is set within a predetermined range. The predetermined range is from 5×10¹ μm to 1×10³ μm. The inscribed sphere is a virtual solid body. The inscribed sphere is preferably in contact with the bottom of each of the compartments 32a and 32b. The size of each of the compartments 32a and 32b is set as described above, thereby promoting the formation of cell aggregates.

The size of each of the droplets 36a and 36b shown in FIG. 3 can be arbitrarily determined as long as the droplets are not broken. The cluster of cells 42a and 42b may be cultured only in the droplets 36a and 36b, respectively. In other words, the cluster of cells 42a and 42b need not necessarily be cultured in the holes 31a and 31b, respectively.

The droplets 36a and 36b shown in FIG. 3 are not necessarily formed. The compartments 32a and 32b may be located in the holes 31a and 31b, respectively. This case corresponds to the case where the bottom openings 34a and 34b are not formed.

The compartments 32a and 32b shown in FIG. 3 are connected to the reservoir compartment 37 through the top portions of the compartments 32a and 32b, that is, the top openings 33a and 33b, respectively. The culture solution 35 in the compartments 32a and 32b is joined with the culture solution 35, which is dispensed in the reservoir compartment 37, through the top portion of each of the compartments 32a and 32b. The cells including the clusters of cells 42a and 42b are not present in the reservoir compartment 37.

As shown in FIG. 3, the structure in which the culture solution in the compartments 32a and 32b is joined with the culture solution in the reservoir compartment 37 has the following advantage. First, the culture solution 35 is moved between the compartments 32a and 32b and the reservoir compartment 37. Therefore, enough nutrients for the clusters of cells 42a and 42b can be supplied, although it is a hanging-drop culture method.

Further, since no cells are present in the reservoir compartment 37 shown in FIG. 3, it is easy to replace the culture solution 35 during the growth of the cell aggregates as described later. A change in pH and/or temperature of the culture solution 35 is less likely to occur because larger amount of the culture solution 35 is supplied than that in the case where the culture solution is dispensed only in the compartments 32a and 32b.

Referring to FIG. 2 again, the culturing device 20 is generally installed in an incubator, but may be moved through the outside air during transporting. The oxygen concentration and temperature in the incubator are different from those in the outside air. Therefore, the culture solution 35 in the culturing device 20 may be affected by the oxygen concentration and temperature of the outside air.

In the conventional hanging-drop method, the reservoir compartment 37 shown in FIG. 2 is not used, so that the oxygen concentration and temperature of the outside air have a great influence on the droplets of the culture solution surrounding the cells. Accordingly, the pH or oxygen concentration of the culture solution rapidly changes. Such a rapid change affects the proliferation and functions of the cells. Further, since it is difficult to replace the culture medium, shortage of nutrients or remaining waste products affect the proliferation and survival of the cells. The culturing device 20 according to this embodiment can alleviate such effects.

The advantageous effect of the culturing device 20 shown in FIG. 2 depends on the plate 30 that forms the reservoir compartment 37. The culture solution 35 in the culturing device 20 is less likely to be affected by a change in the external environment. Therefore, the effects on the cell aggregates formed by the clusters of cells can be reduced.

### [Bringing Clusters of Cells Close to Each Other]

The clusters of cells separated from each other in the population 41 shown in FIG. 3 are distributed into the compartments 32a and 32b, with the result that the clusters of cells are brought close to each other. As described above, the holes 31a and 31b are gradually narrowed toward the bottom portion of the plate 30 from the top portion thereof, which promotes the operation of bringing the clusters of cells close to each other. By bringing the clusters of cells close to each other, the clusters of cells can be efficiently clumped or assembled.

### [Clumping or Assembling of Clusters of Cells]

In step 22 shown in FIG. 1, two or more clusters of cells are clumped or assembled in each of the compartments 32a and 32b shown in FIG. 3. In the first example, two or more clusters of cells including a cluster of cells 42a are clumped or assembled in the compartment 32a. In the other example, two or more clusters of cells including a cluster of cells 42b are clumped or assembled in the compartment 32b.

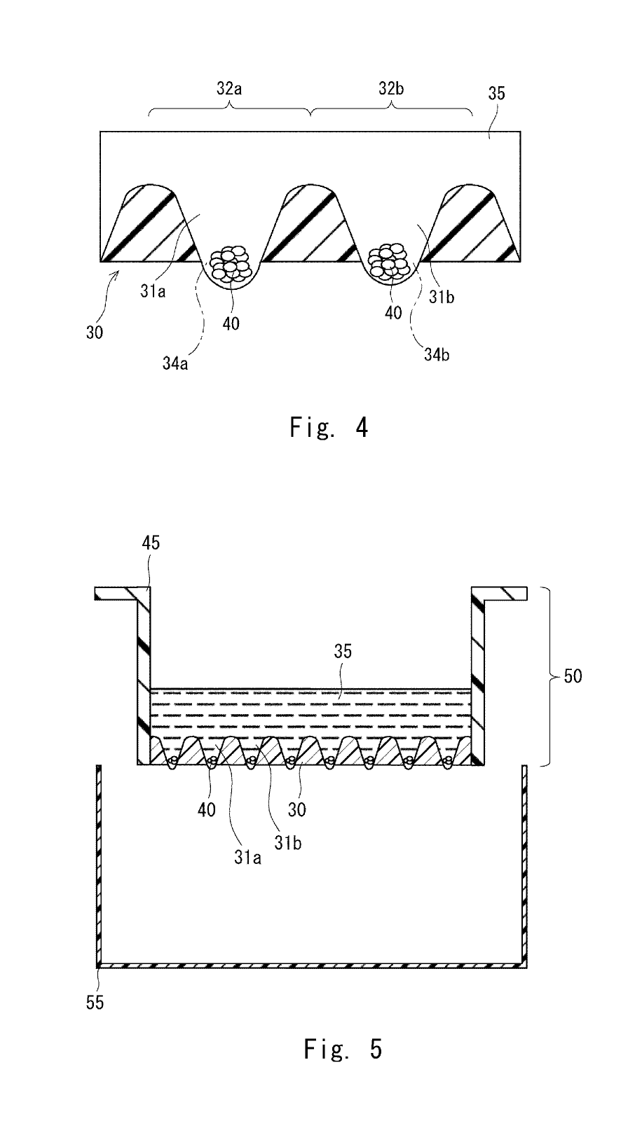

FIG. 4 is an enlarged sectional view of cell aggregates 40 and the plate 30. As a result of clumping or assembling of the clusters of cells, the cell aggregates 40 are formed in the compartments 32a and 32b, respectively.

The two or more clusters of cells including the cluster of cells 42c shown in FIG. 3 are also clumped or assembled in any one of the compartments. The sizes of the clusters of cells distributed into the compartments are substantially non-uniform. Accordingly, a population of clusters of cells having non-uniform sizes is clumped or assembled in each of the compartments. For example, a population of clusters of cells including the clusters of cells 42a to 42c may be clumped or assembled in each of the compartments.

As described above, in step 21 shown in FIG. 1, the suspension 38 is spread to distribute the populations 41 into the compartments as shown in FIG. 3. After distribution, the clusters of cells are clumped or assembled. Therefore, the non-uniformity of the size of clusters of cells before being clumped or assembled can be relieved. Furthermore, the distribution states of the sizes of clusters of cells in each compartment can be normalized. Accordingly, as shown in FIG. 4, the cell aggregates 40 are formed in the respective compartments through the clumping or assembling of the clusters of cells and the cell aggregates 40 obtain homogeneity. Further, the cell aggregates 40 can be formed to have a uniform size.

Before the clusters of cells are distributed into the compartments 32a and 32b shown in FIG. 3, the clusters of cells are separated from each other. Then, after the clusters of cells are distributed into the compartments, the clusters of cells are brought close to each other. Therefore, clumping or assembling of the clusters of cells is started when the distribution is complete. Prior to the distribution, pipetting is suitably performed to separate the clusters of cells and mix the clusters of cells. Other methods may also be used.

### [Growth of Cell Aggregates and Recovery of Cell Aggregates]

In step 23 shown in FIG. 1, the cell aggregates 40 shown in FIG. 2 are allowed to grow. Step 23 is carried out before the decomposition of the cell aggregates shown in step 25. FIG. 4 is an enlarged view showing the formed cell aggregates and the plate 30. In the process of growth in the compartments 32a and 32b, the cell aggregates 40 are allowed to grow. The cell aggregates are formed by steps 23 and 24.

In the process of forming the cell aggregates, steps 22 and 23 shown in FIG. 1 may be carried out simultaneously in the compartments 32a and 32b shown in FIG. 3. In the process of growing of the clusters of cells 42a and 42b, the clusters of cells may be clumped or assembled to form the cell aggregates 40 shown in FIG. 4. The cell aggregates 40 may be allowed to grow after the clusters of cells 42a and 42b are rapidly assembled or assembled to form the cell aggregates 40.

In step 23 shown in FIG. 1, the cell aggregates 40 shown in FIG. 4 are preferably allowed to grow for a period of from 2 to 14 days. This period is preferably three to seven days. At the time when the diameter of each cell aggregate is equal to or less than a predetermined value, it is preferable to stop growing cell aggregates and recover the cell aggregates as shown in step 24.

The diameter of the cell aggregates including the cell aggregates 40 shown in FIG. 4 indicates the diameter of a circumscribed sphere of each cell aggregate. A predetermined value of the diameter of each cell aggregate is ¾ or less of the diameter of each of the bottom openings 34a and 34b, and preferably, ⅔ or less of the diameter of each of the bottom openings 34a and 34b.

The predetermined value of the diameter of each cell aggregate is preferably one of the values of 1 mm, 0.9 mm, 0.8 mm, 0.7 mm, 0.6 mm, 0.5 mm, 0.4 mm, 0.3 mm, 0.2 mm, and 0.1 mm. As described above, the recovery of the cell aggregates makes it possible to prevent the cell aggregates from growing into extremely large cell aggregates and prevent contamination of cell aggregates inside of which differentiation occurs.

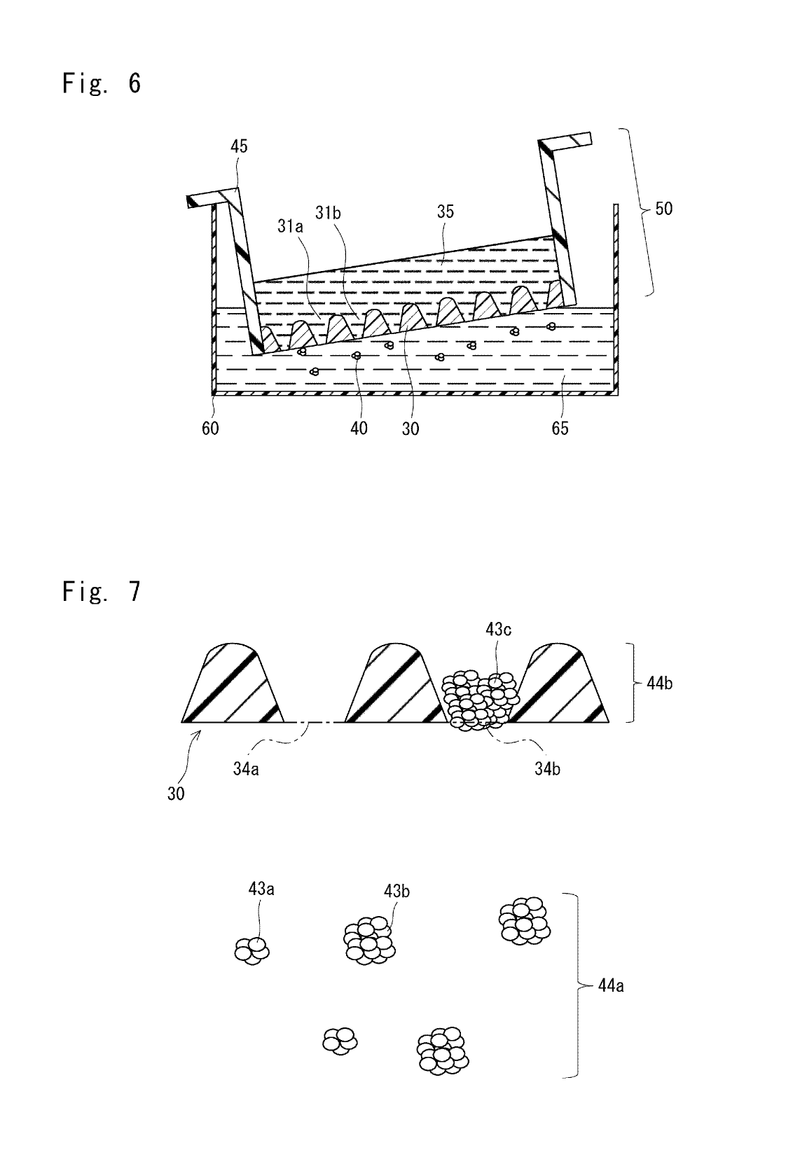

In step 24 shown in FIG. 1, the chamber 50 and the tray 55 are first separated from each other as shown in FIG. 5. Next, as shown in FIG. 6, the bottom of the plate 30 is soaked in a recovery solution 65 in a tray 60. The tray 60 may have the same structure as that of the tray 55.

As shown in FIG. 6, the cell aggregates 40 are allowed to pass through the bottom of the plate 30. The cell aggregates 40 are moved from the culture solution 35 to the recovery solution 65, or the culture solution 35 and the cell aggregates 40 flow into the tray 60. This process may be carried out using the gravity or by suction. Through this process, the cell aggregates 40 are separated from the plate 30. Thus, the cell aggregates 40 are recovered into the recovery solution 65. The recovery solution 65 may be a culture medium or buffer solution.

As described above, in the case where the holes 31a and 31b shown in FIGS. 5 and 6 are recesses, the cell aggregates cannot be caused to pass through the bottom of the plate 30. In this case, the cell aggregates 40 may be recovered by pipetting. The method of causing the cell aggregates 40 to pass through the bottom of the plate 30 is advantageous in providing little physical stimulation to the cell aggregates 40. In this method, the undifferentiated state of cell aggregates is less likely to be impaired.

FIG. 7 is an enlarged view showing the state in which the chamber and the cell aggregates are separated. Cell aggregates 43a to 43c are obtained by classifying the cell aggregates 40 according to the size of the cell aggregates. The cell aggregate 43a is smaller than the cell aggregate 43b. The cell aggregate 43c is larger than the cell aggregate 43b.

As shown in FIG. 7, the diameter of each of the cell aggregates 43a and 43b is smaller than the diameter of the bottom opening 34a. Accordingly, the cell aggregates 43a and 43b pass through the bottom opening 34a. Through the above-mentioned separation process, a population 44a of cell aggregates is obtained.

The diameter of the cell aggregate 43c is larger than the diameter of the bottom opening 34b. Accordingly, the cell aggregate 43c does not pass through the bottom openings 34a and 34b. Through the above-mentioned separation process, a population 44b of cell aggregates is left in the plate 30.

The population of the cell aggregates 40 shown in FIG. 5 is separated into the population 44a and the population 44b shown in FIG. 7 due to the operation of the plate 30 shown in FIG. 6. In other words, the plate 30 has a filter function.

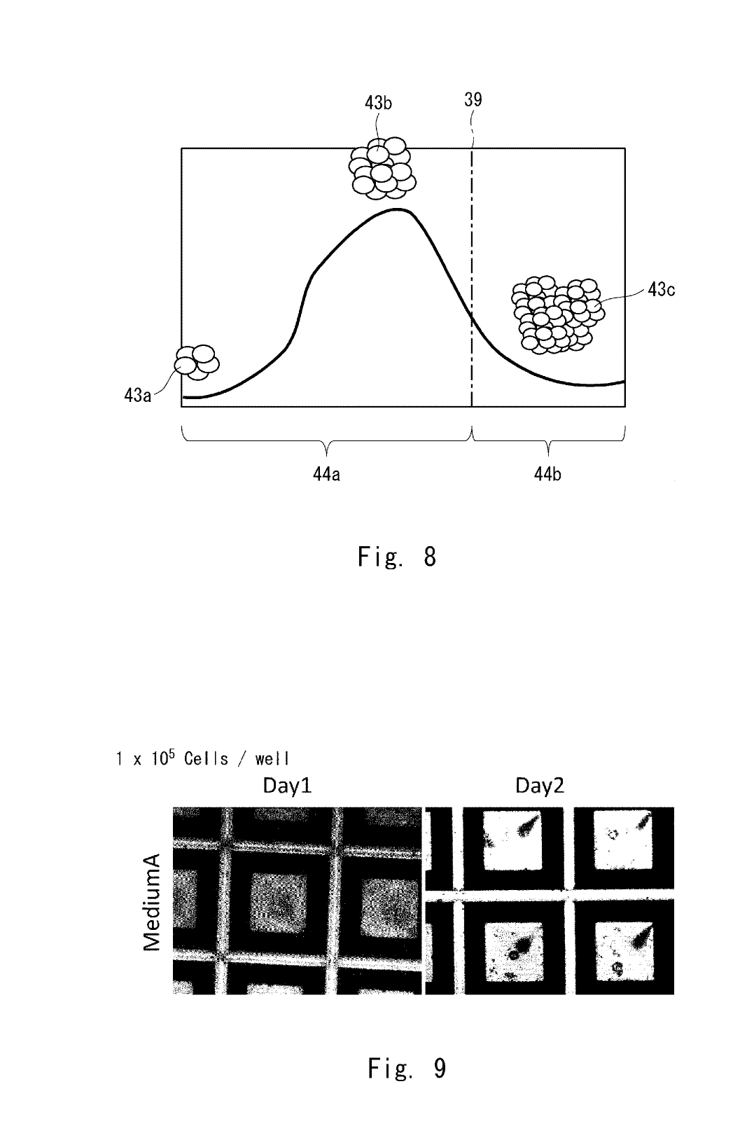

FIG. 8 is a graph showing a distribution of sizes of cell aggregates. The horizontal axis represents the size of each cell aggregate. The vertical axis represents the number of cell aggregates as a ratio. The size of each of the cell aggregates 43a and 43b included in the population 44a is smaller than a threshold 39. The size of the cell aggregate 43c included in the population 44b is larger than the threshold 39 of each of the bottom openings 34a and 34b.

The threshold 39 shown in FIG. 8 depends on the diameter of each of the bottom openings 34a and 34b. The threshold 39 is equal to the diameter of each of the bottom openings 34a and 34b. As shown in FIG. 7, the size of each of the cell aggregates 43a and 43b which are separated from the plate 30 can be controlled by the diameter of each of the bottom openings 34a and 34b. The plate 30 filters the cell aggregates according to the threshold 39.

The diameter of each of the recovered cell aggregates 43a and 43b shown in FIG. 7 is preferably 1 mm or less as described above. The cell aggregates having the diameter can be achieved by adjusting, for example, the growth period or growth conditions. The above-mentioned filter function makes it possible to select the cell aggregates 43a and 43b having the diameter. It is expected that the filter function of the plate 30 provides the following preferable effects.

When cells that proliferate more rapidly than cells with normal proliferation rate are included in the clusters of cells 42a and 42b shown in FIG. 3, the cell aggregates 40 (FIG. 4) obtained by clumping or assembling the clusters of cells may be larger than normal cases. A change in the rate of proliferation is caused by, for example, karyotype abnormalities in the cells.

Cells with karyotype abnormalities proliferate more rapidly than normal cells, and the survival rate of the cells with karyotype abnormalities is higher than that of normal cells. Therefore, even when the clusters of cells having the same size are allowed to grow for the same period of time, the clusters of cells including cells with karyotype abnormalities grow into cell aggregates larger than that of normal clusters of cells. A rate of appearance of such cell aggregates is not negligible.

Cells with karyotype abnormalities are preferably not included in the cell aggregates. This is because the cell aggregates may be used for various tests, medical treatments, and the like, and thus it is preferable that the cell aggregates exhibit normal functions. On the other hand, even when the growth period and growth conditions are adjusted, karyotype abnormalities may occur with a certain probability.

The filter function of the plate 30 shown in FIG. 7 makes it possible to remove the cell aggregate 43c from the population 44a. For example, it can be assumed that the cell aggregate 43c has grown into a cell aggregate larger than a normal cell aggregate due to the karyotype abnormalities. Accordingly, the cell aggregate with karyotype abnormalities can be removed from the population 44a by the filter function of the plate 30.

To obtain the above-mentioned advantageous effects, the diameter of each of the bottom openings 34a and 34b shown in FIG. 7 is preferably one of the values of 1.0 mm, 0.9 mm, 0.8 mm, 0.7 mm, 0.6 mm, 0.5 mm, 0.4 mm, 0.3 mm, 0.2 mm, and 0.1 mm.

The bottom openings 34a and 34b shown in FIG. 7 preferably have an equal inscribed circle diameter. Not only the bottom openings 34a and 34b, but also a plurality of bottom openings of the holes, preferably, all the bottom openings, have an equal inscribed circle diameter. When the bottom openings have an equal inscribed circle diameter, the cell aggregates to be recovered can be formed with a uniform upper limit size. Further, the bottom openings preferably have an equal area.

### [Decomposition of Cell Aggregates and Mixing of Clusters of Cells]

In step 25 shown in FIG. 1, the recovered cell aggregates are broken up. At the time when the diameter of each cell aggregate is 1 mm or less, the cell aggregates are preferably broken up. Thus, the contamination of differentiated cells in the cell aggregates can be prevented, during the number of cell aggregates is increased as described later. In other words, a uniform undifferentiated state can be maintained among the cell aggregates.

The cell aggregates to be broken up are cell aggregates included in the recovered population 44a as shown in FIG. 7. The cell aggregates are broken up to generate a plurality of clusters of cells. The decomposition may be carried out by physical dissociation of the cell aggregates. The physical dissociation may be carried out by pipetting. The decomposition may be carried out by an enzyme treatment. The cell aggregates subjected to the enzyme treatment may be physically dissociated. The enzyme treatment may be performed on the physically dissociated cell aggregates to thereby generate clusters of cells.

In step 26 shown in FIG. 1, the clusters of cells are further mixed with each other. The mixture of clusters of cells is generated from cell aggregates different from each other. The mixing of clusters of cells may be performed by pipetting. When the dissociation is performed by pipetting, the mixing of clusters of cells can be performed at the same time.

### [Cycle of Steps]

Referring again to step 21 shown in FIG. 1, the populations 41 of the mixed clusters of cells is distributed into the compartments as typified by the two or more compartments 32a and 32b as shown in FIG. 3. Two or more mixed clusters of cells are distributed to each of the two or more compartments. A newly prepared culturing device is preferably used as the culturing device shown in FIG. 2.

In step 22 shown in FIG. 1, the distributed clusters of cells are brought close to each other again in the compartments 32a and 32b. Two or more clusters of cells brought close to each other are clumped or assembled again. Specifically, the steps are executed in the order of (clumping or assembling)->(decomposition)->(clumping or assembling). After the mixed clusters of cells are distributed into the compartments, the clusters of cells are clumped or assembled again, thereby making it possible to further increase the number of cell aggregates having homogeneity among the cell aggregates while maintaining the homogeneity among the cell aggregates.

In the flowchart shown in FIG. 1, there is no limitation on the number of steps to return from step 26 to step 21. Accordingly, the described process of breaking up the increased cell aggregates, mixing the clusters of cells obtained after breaking up the cell aggregates, distributing the cell aggregates, bringing the cell aggregates close to each other, and clumping or assembling the cell aggregates again may be repeated once or twice or more.

In the method described above, the steps are repeatedly performed in the order of (clumping or assembling)->(decomposition)->(clumping or assembling)->(decomposition)->(clumping or assembling)->. . . . As a result, the number of cell aggregates having homogeneity among the cell aggregates can be increased while maintaining the homogeneity among the cell aggregates.

As indicated by an arrow 27 shown in FIG. 1, step 25 may be omitted in an arbitrary cycle. In this case, as described above, the cell aggregates formed by clumping or assembling the cell aggregates again in step 22 are not broken up in step 25. Accordingly, the process shifts from step 24 to step 26 and the cell aggregates are mixed with each other.

Through the process as indicated by the arrow 27 shown in FIG. 1, the process returns to step 21 from step 26. The cell aggregates mixed in step 26 are distributed into the two or more compartments 32a and 32b as if they were the clusters of cells 42a to 42c shown in FIG. 3. The two or more cell aggregates mixed in the compartments are brought close to each other. In step 22, the two or more cell aggregates brought close to each other are clumped or assembled in each of the compartments 32a and 32b.

In the method described above, the steps are executed in the order of, for example, (clumping or assembling)->(decomposition)->(clumping or assembling)->(clumping or assembling). This method makes it possible to increase the size of cell aggregates having homogeneity among the cell aggregates, while suppressing a deterioration in the homogeneity.

For example, step 25 shown in FIG. 1 may be omitted. In step 26, the different cell aggregates formed are mixed with each other. The process returns to step 21 and the mixed cell aggregates are distributed into each of two or more compartments. The two or more cell aggregates mixed in each of the compartments are brought close to each other. In step 22, the two or more cell aggregates brought close to each other are further clumped or assembled in each of the compartments.

In the above-described method, the steps are executed in the order of (clumping or assembling)->(clumping or assembling). This method makes it possible to further increase the size of each of the cell aggregates having homogeneity among the cell aggregates, while suppressing a deterioration in the homogeneity.

For example, as described above, the cell aggregates which are formed into large cell aggregates without carrying out step 25 shown in FIG. 1 may be broken up in step 25. Specifically, as described above, the cell aggregates formed by further clumping or assembling the cell aggregates in step 22 are broken up in step 25. The clusters of cells are generated from the cell aggregates which are formed into large cell aggregates.

In step 26 shown in FIG. 1, the clusters of cells generated from different cell aggregates are mixed with each other. The process returns to step 21, and the mixed clusters of cells are distributed into each of two or more compai invents. The two or more cell aggregates mixed in each of the compartments are brought close to each other. In step 22, the two or more clusters of cells brought close to each other are clumped or assembled again in each of the compartments. In the above-described method, the steps are executed in the order of, for example, (clumping or assembling)->(clumping or assembling)->(decomposition)->(clumping or assembling).

### [Initial Preparation of Clusters of Cells]

In step 21 shown in FIG. 1, clusters of cells from which cell aggregates are formed may be prepared by any method. For example, pluripotent stem cells may be cultured in a plate to form a colony. According to step 25, the colony is broken up to generate clusters of cells. According to step 26, the clusters of cells are mixed with each other.

A population of the clusters of cells is used as the population 41 shown in FIG. 3 for the distribution in step 21 (FIG. 1) of a first cycle. This method makes it possible to obtain cell aggregates, which are homogenized in the cell aggregates, from the cells cultured in a plate.

As described above, when the colony is broken up, pipetting may be performed. The colony may be broken up only by an enzyme treatment. Only physical dissociation may be performed. Both the enzyme treatment and physical dissociation may be performed.

### [Use of Cell Aggregates]

The cell aggregates obtained as described above may be cultured by suspension culture or adherent culture. In the culture, the pluripotent stem cells in the cell aggregates may be differentiated in accordance with a predetermined method. Examples of the predetermined method to be employed include an in vitro differentiation-inducing system.

In this embodiment, cell aggregates are obtained as a population. In this embodiment, the sizes of the cell aggregates of pluripotent stem cells collected in each cycle are equalized in the entire process. Therefore, in the population, a uniform undifferentiated state is maintained in the cell aggregates. Therefore, the cell aggregate according to this embodiment is suitably used to maintain a uniform differentiated state in the pluripotent stem cells when the pluripotent stem cells are differentiated as described above.

Whether the undifferentiated state of the population is maintained or not can be determined by a positive rate of a pluripotent stem cell marker. For example, it is only necessary that 80% or more of all cell aggregates of the population of cell aggregates are positive. The positive rate in the population of cell aggregates is calculated as a ratio of cell aggregates for which the pluripotent stem cell marker is positive.

For example, if the ratio of the cell aggregates for which the pluripotent stem cell marker is positive in a population of cell aggregates is 80% or more, it may be determined that the undifferentiated state of the population is maintained.

The measurement can be performed by the following method. First, ten stem cell aggregates are selected from the population of cell aggregates. In each of the selected cell aggregates, 100 cells are selected. The selected cells may be 100 or more. It is determined whether the pluripotent stem cell marker is positive for the 100 cells, and the positive rate in one cell aggregate is measured. In the determination, if the pluripotent stem cell marker is positive for three or more cells among the 100 cells, it is determined that the pluripotent stem cell marker is positive for the cell aggregate. Note that in the determination, when 1,000 or more cells are selected, if 3% or more of the cells are positive, it is determined that the pluripotent stem cell marker is positive for the cell aggregate.

By the method described above, the ratio (positive rate) of the cell aggregates for which the pluripotent stem cell marker is positive among 10 cell aggregates is obtained. The measurement of the positive rate is further performed on the same population twice. That is, the measurement of the positive rate is performed three times in total. The average of the positive rates obtained by performing the measurement three times is used as an average value of the positive rates.

For example, TRA-1-60 may be used as the pluripotent stem cell marker. Whether TRA-1-60 is positive or not can be determined based on whether or not a positive cell population appears, in comparison with a negative cell population, by using, for example, a flow cytometer. As another method, the pluripotent stem cell marker may be detected by the PCR. In this case, at least one of Nanog and Oct3/4 may be selected as the pluripotent stem cell marker. Marker gene expressions are detected by controlling differentiated cells, such as fibroblast cells, which are not expressed.

In the population of cell aggregates, the cell aggregates preferably have homogeneous functions. Whether the cell aggregates have homogeneous functions can be determined by an in-vivo induced differentiation method, such as a capability of forming a teratoma. When a cell aggregate or a pluripotent stem cell in the cell aggregate is implanted in a mouse, it can be determined whether a teratoma is formed in the body of the mouse. In the cell aggregates of the population, the ratio of the cell aggregates forming teratoma differentiated into three germ layers is preferably 80% or more, more preferably, 95% or more, and most preferably, 100%. In this case, it can be determined that the functions in the population are homogeneous.

In the population of cell aggregates, the uniformity of the differentiation capability is preferably maintained in the cell aggregates. Whether the differentiation capability is homogeneous can be determined based on whether the cells in the cell aggregates are differentiated into three germ layers when the cell aggregates are induced-differentiated.

For example, ten cell aggregates are selected from the population. Ten or more cell aggregates may be selected. The differentiation into any one of three germ layers in vivo is induced for each of the ten cell aggregates. In another aspect, the differentiation of the cell aggregates is induced to form an embryoid body. The term “embryoid body” described herein refers to a cell aggregate of cells including various differentiated cells, such as a fertilized egg or an embryo. In each cell aggregate, 80% or more of formed embryoid bodies preferably express a germ layer marker in any one of three germ layers. It is preferable that all the 10 or more selected cell aggregates satisfy the requirement.

The gene expression level of each embryoid body may be determined by measurement using a PCR method.

In another aspect, the ratio of embryoid bodies induced from each cell aggregation by the in vitro differentiation-inducing system is preferably 80% or more. It is preferable that all the 10 or more selected cell aggregates satisfy the requirement. The term “embryoid body” described herein refers to a cell aggregate in which tissues of three germ layers are mixed.

The differentiation marker is preferably at least one of ectoderm, endoderm, and mesoderm differentiation markers. For the ectoderm differentiation marker, at least one of Pax6, SOX2, PsANCAM, and TUJ1 may be used. For the endoderm differentiation marker, at least one of FOXA2, AFP, cytokine 8.18, and SOX17 may be used. For the mesoderm differentiation marker, at least one of Brachyury and MSX1 may be used.

Note that the present invention is not limited to the above-described embodiments and below-shown examples and can be modified as appropriate without departing from the scope of the invention. For example, in the above embodiments, after cell aggregates are formed from clusters of cells, the cell aggregates are recovered. However, after clusters of cells are formed by clumping or assembling two or more clusters of cells, the large clusters of cells may be recovered. The large clusters of cells need not necessarily grow into the above-mentioned size of cell aggregates. In other words, cell aggregates having a sufficient size and function may be finally obtained by repeating the above-mentioned cycle. Further, as another embodiment according to the present invention is a cell culture method for pluripotent stem cells. In this embodiment, similar to the above-described embodiments, cells may be made to grow in order to increase pluripotent stem cells or maintain the survival of pluripotent stem cells.

### EXAMPLES

### Example 1

### <Acquisition of iPS Cells>

As pluripotent stem cells, induced pluripotent stem cells (iPS cells) in which an un-differentiation marker Nanog, Oct3/4, or TRA1-60, or an un-differentiation marker similar thereto was expressed, and which had been confirmed to be differentiated into three germ layers were used.

### (Cell Culturing)

The above-described iPS cells were used to form clusters of cells from which cell aggregates were formed. Firstly, the iPS cells were cultured on feeder cells for five to seven days in a 6-well plate. After confirming that the iPS cells became 70 to 80% confluent, the medium was removed from the well by using an aspirator. For each well, 500 μL of a Dissociation Solution for human ES/iPS Cells (CTK solution, ReproCELL Inc.) was added in the well. The 6-well plate was incubated for three minutes in a CO₂ incubator (37° C., 5% CO₂).

After the incubation, the 6-well plate was brought out from the CO2 incubator. The feeder cells were peeled off by tapping the well plate or the wells. After that, the CTK solution was removed by an aspirator and 1 ml of PBS was added in each well.

A microscope was used to confirm that the feeder cells on the 6-well plate were peeled off After that, the PBS was removed from the dish by an aspirator. After that, 500 μl of TrypLE Select Enzyme (1×) (Trademark; manufactured by Thermo Fisher Scientific; hereinafter referred to as “TrypLE Select”) was added in each well and then the well plate was incubated for five minutes in the CO₂ incubator.

A medium Y was manufactured as a culture medium for ES cells or iPS cells as follows. Firstly, a Human ES medium (reprocell Inc.) was prepared as a basic medium. Further, 0.2 ml of a 10 μg/ml basic fibroblast growth factor (bFGF) (Thermofisher PHG0266) was added in the above-described medium.

After incubation, the 6-well plate was removed from the CO₂ incubator. 500 μl per well of medium was added into the well. By using a Pipetman (P1000), iPS cells were suspended 10 to 30 times. Those suspending actions were carried out in a similar manner also in Example 3 and the subsequent examples. Through the above-described processes, a suspension containing a population of clusters of cells was prepared. This suspension also contained single cells of iPS cells formed by the suspending action. The medium was replaced and the population of clusters of cells was eventually suspended in a commercially-available feeder-free culture solution. In this Example, the feeder-free culture solution is referred to as a culture solution A (a Medium A).

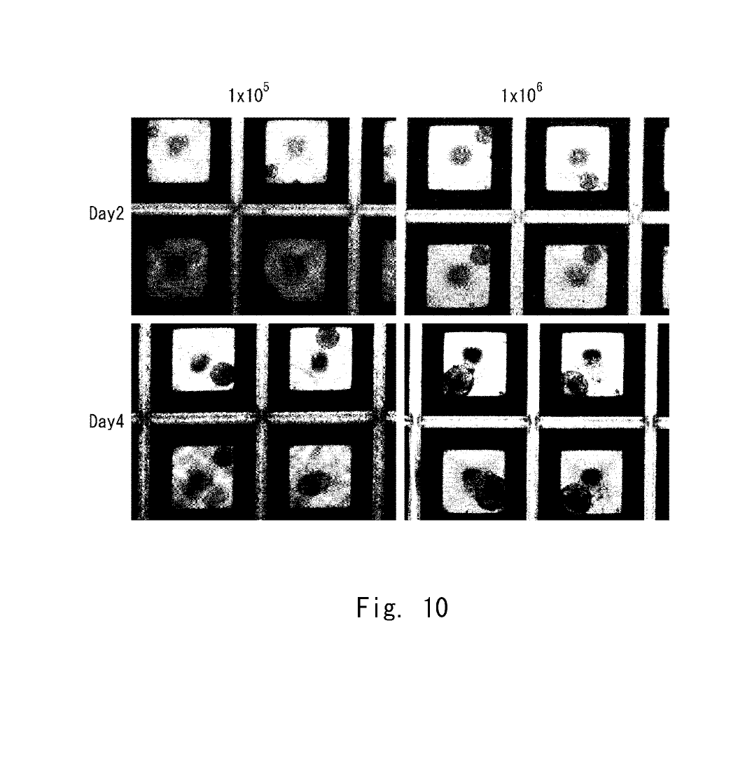

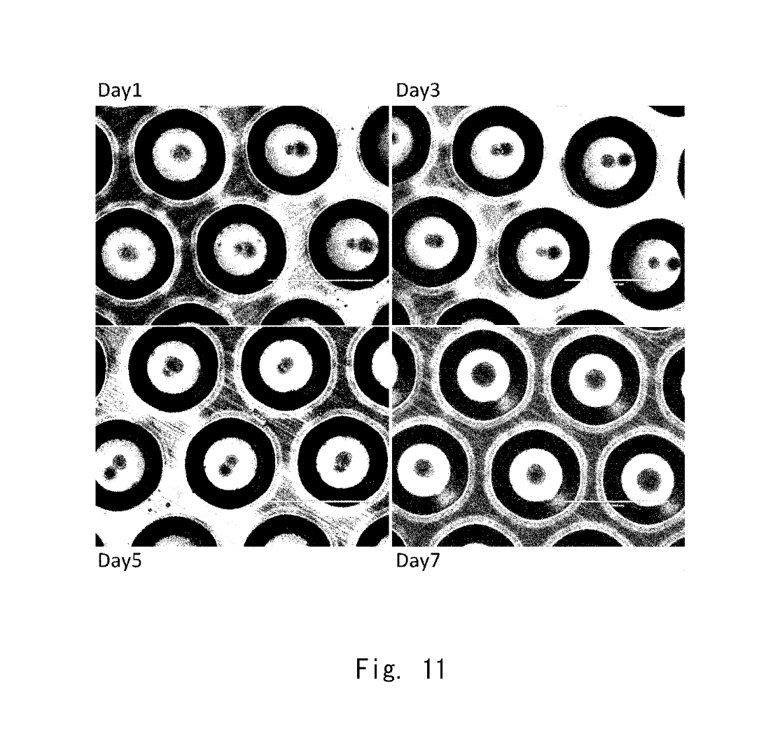

As a plate for forming cell aggregates (hereinafter referred to as a plate, unless otherwise specified), a <Elplasia> plate manufactured by Kuraray Co., Ltd. was used. Among the <Elplasia> plates, a Multiple Pore Type plate was used. As shown in FIG. 9, the Multiple Pore Type plate includes a plurality of wells that are formed in the form of through-holes.

FIG. 9 shows observation images of cell aggregates when the plate is viewed from above. As shown in FIG. 9, the wells have the same sizes as each other. A top opening and a bottom opening of a through hole are both rectangular. Specifically, the top opening and the bottom opening are both square. Directions of corners of the top and bottom openings are aligned with each other when these openings are viewed from above. Further, these openings are concentric.

The length of one side of the top opening is 650 μm. The length of one side of the bottom opening is 500 μm. In the plate, 680 wells are arranged in an orderly manner on the bottom surface having an area of 7 cm². That is, the number N of compartments formed by the wells is 680 (N=680). Specifically, the wells are arranged in a square lattice pattern. A unit of the lattice is a square 500 μm on each side.

The culture solution was uniformly sown over the entire surface of the plate so that at least two clusters of cells were distributed to each of the compartments formed by the respective wells. The culture solution was spread over all the wells. Clusters of cells were distributed so that 1×10⁵ cells were contained in each compartment (the number n of cells in each compartment is 1×10⁵ (n=1×10⁵)). It is presumed that since the sizes of the top openings are equal to each other and the wells are uniformly arranged in the lattice pattern, the numbers of cells distributed to the compartments are equal to each other.

The number of cells per unit volume in the culture solution A, i.e., a cell concentration C [1/ml] was determined according to a formula C=N·n/V. In the formula, N represents the number of compartments and n represents the number of cells in each compartment. Further, V represents the volume of the culture solution A used in one plate.

A droplet of the culture solution A protruded from the bottom opening of each well (FIG. 3). A meniscus, which is an interface between the droplet and the atmosphere, was formed by the surface tension of the culture solution A. Since clusters of cells gathered toward the meniscus, the clusters of cells moved close to each other in the compartment formed by the inner surface of the well and the meniscus. In this manner, the clusters of cells were cultured in the culture solution A dispensed in the compartment. Note that in this Example, as described previously, the meniscus is also regarded as a part of the components of the compartment.

As shown in Day 1 and Day 2 of the Medium A shown in FIG. 9, the clusters of cells, which had been moved close to each other, were clumped or assembled. Day 1 and Day 2 indicate that one day and two days, respectively, have elapsed since the seeding. In the other figures, a number next to “Day” or “day” represents the number of days that have elapsed since the date of the first seeding on the plate. As described above, cell aggregates of pluripotent stem cells were obtained by making cells grow while clumping or assembling clusters of cells.

### Example 2

In Example 1, the feeder cells and the iPS cells were peeled off from the wells by using the Dissociation Solution for human ES/iPS Cells. Further, the iPS cells were treated by using the TrypLE Select Enzyme.

In contrast to this, in Example 2, iPS cells were scraped off by using a scraper, but the enzyme treatment was not carried out. The number of times that the iPS cells were suspended was less than 10 times. The rest of the processes were similar to those in Example 1.

In Example 2, cells forming clusters of cells account for 80% of cells contained in the suspension that was spread on the plate to be formed to cell aggregates.

### Example 3

In this Example, cells were cultured in a manner similar to that in the above-described Example 1, except that the number n of cells distributed to one compartment was changed to 1×10⁶. FIG. 10 shows observation images of cell aggregates when the plate is viewed from above. Right side of FIG. 10 shows the results of the case of distributing 1×10⁶ cells to each compartment. The left side shows the results of the comparison case of distributing 10⁵ cells to each compartment as the case of the previous Example. FIG. 10 shows results on the second and fourth days after the seeding.

It has been found that the number of cells distributed in a range from 1×10⁵ to 1×10⁶ per compartment allows to obtain a cell aggregates having uniform sizes.

### Example 4

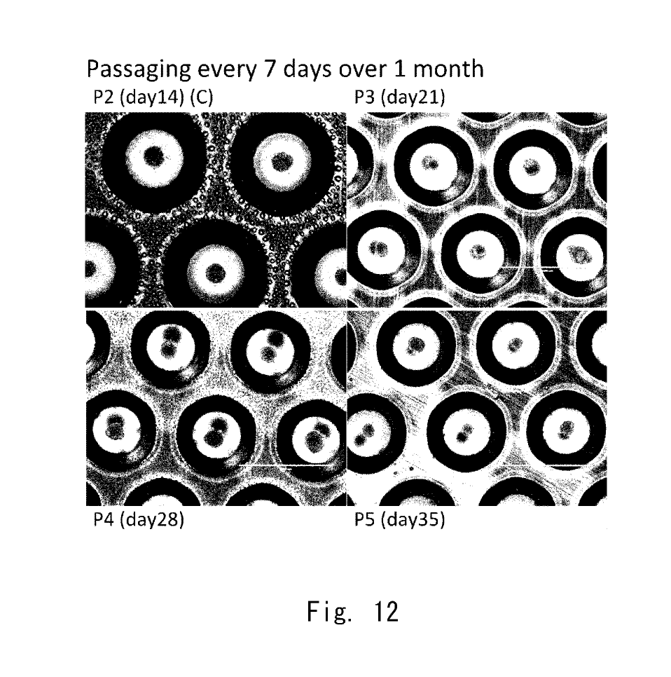

FIG. 11 shows observation images of cell aggregates when the plate is viewed from above. In this Example, the shapes of top and bottom openings were circular as shown in FIG. 11. These openings are concentric when the plate is viewed from above. The length of bars in the cross direction shown in the observation images represents 1,000 μm. The iPS cells were cultured under conditions similar to those in Example 1, except for the above-described matter.

The diameter of the top opening is 650 μm. The diameter of the bottom opening is 500 μm. In the plate, 648 wells are arranged in an orderly manner on the bottom surface having an area of 7 cm².

FIG. 11 shows cell aggregates on the first, third, fifth and seventh days after clusters of cells were sown. On each day, cell aggregates having uniform sizes were obtained over all the compartments. Generally, one cell aggregate was obtained in each compartment. There was no significant difference among the rates at which the sizes of cell aggregates in the respective compartments increase with the lapse of time. Therefore, it has been indicated that the quality of cells was uniformly maintained over all the compartments.

It is expected that the number of cells in each compartment is no less than 2,000 and no more than 5,000 on the seventh day.

### Example 5

Cells were cultured in a manner similar to that in Example 4, unless otherwise specified. Cell aggregates obtained on the seventh day in the culturing were recovered from the plate. In the recovery process, the cell aggregates were made to pass through the bottom opening. Specifically, as shown in FIG. 6, they were recovered by bringing the bottom surface of the plate into contact with a recovery solution and thereby eliminating the interface of the culture solution. Hereinafter, this recovery method is referred to as a contact method. The recovery solution was recovered in a 15 ml tube.

After centrifuging the tube at 270 G, a supernatant was removed. Then, 500 μ1 of TrypLE Select was added in the tube and the tube was incubated in an incubator at 37° for ten minutes. After centrifugation, a supernatant was removed and cells in the tube were suspended in 1 ml of the Medium A. The number of suspended cells was counted by using a hemocytometer. Based on the calculated number of cells, 2×10⁵ iPS cells were suspended in 2 ml of the Medium A. Further, after 2 μl of a ROCK (Rho-associated coiled-coil forming kinase/Rho-associated kinase) inhibitor (a ROCK Inhibitor) was added, the iPS cells were sown on the plate. During the culturing, the old medium was replaced by Medium A supplemented with 1 μl of the ROCK Inhibitor per 1 ml of the Medium A. The medium replacement was carried out every day.

A culture solution was sown again on a plate having the same shape. The culture solution was spread over all the wells. At least two mixed clusters of cells were distributed to each compartment, at least two compartments. The number of cells in each compartment was roughly the same as that of the first seeding. Clusters of cells were moved close to each other in each compartment. In this way, clusters of cells were clumped or assembled again.

Passage was carried out by repeating steps of breaking up cell aggregates and thereby obtaining clusters of cells, mixing the clusters of cells, moving them close to each other, distributing them, and clumping or assembling them again. The passage was repeated twice (P2), three times (P3), four times (P4), and five times (P5). Regarding how to count the number of times of passage, passage in which clusters of cells obtained from an iPSC colony are sown in a plate for the first time is regarded as first passage (P1).

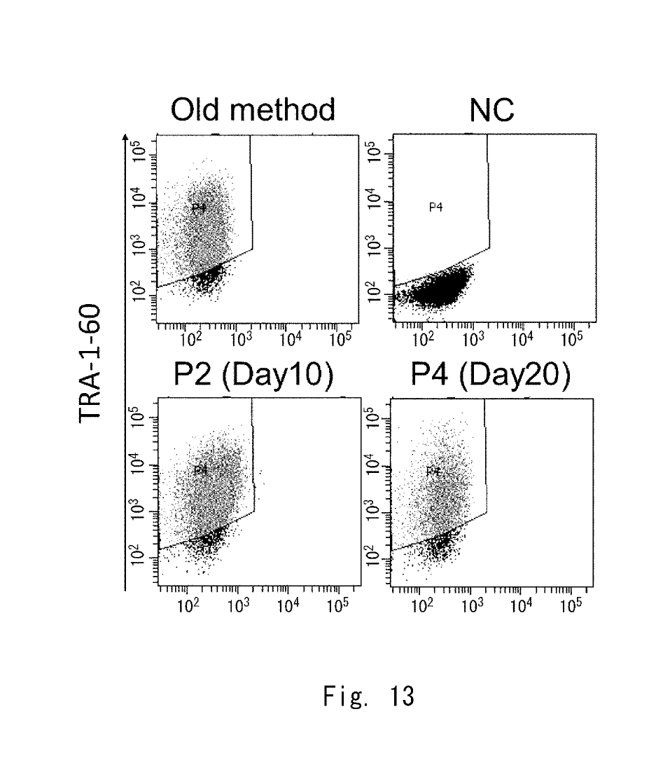

The passage was carried out every seven days. FIG. 12 shows observation images of cell aggregates on the 14th, 21st, 28th and 35th days after the first seeding on the plate. The length of bars in the cross direction shown in the observation images represents 1,000 μm. Even after a long period of one month, cell aggregates having uniform sizes were obtained over all the compartments. After the long-term culturing, pluripotent stem cells were evaluated by flow cytometry.

<Flow Cytometry and Fluorescence Activated Cell Sorting (FACS)>

Cell aggregates obtained on the 10th and 20th days in the culturing were recovered by the above-described contact method and collected in a 15 ml tube. After centrifuging the tube at 270 G, a supernatant was sucked out. Cells were individualized by adding 500 μl of TrypLE Select in the tube and incubating the tube in an incubator at 37° for ten minutes. After incubating the tube for ten minutes, 500 μl of the Medium A was added in the tube. Cell aggregates were dissociated by suspending action for the cell aggregates and the Medium A repeating 10 to 30 times by using a Pipetman. After adding 9 ml of the Medium A in the tube, the tube was further centrifuged at 270 G.

After the centrifugation, a supernatant was sucked out from the tube and precipitated cells were suspended in 1 ml of the Medium A. The number of suspended cells was counted by using a hemocytometer. Based on the calculated number of cells, 1×10⁶ cells were injected into each of new tubes. The tube was centrifuged at 270 G again. After the centrifugation, a supernatant in the tube was sucked out. Then, 2.5 μl of an antibody for detecting TRA-1-60 was suspended in 50 μl of PBS. After this antibody suspension was added in the tube, the tube was incubated at a room temperature for 30 minutes under a light-shielded condition.

After 30 minutes had elapsed, 1 ml of PBS was added in the tube. After centrifuging the tube at 270 G, a supernatant was removed from the tube. A positive rate of TRA-1-60 of iPS cells was measured by using a flow cytometer Cytoflex.

FIG. 13 shows four FACS histograms. The vertical axis in the histogram indicates the strength of TRA-1-60. The horizontal axis indicates the intensity of intrinsic fluorescence.

The upper-left histogram (Old method) in FIG. 13 shows a result of positive control. For the positive control, subculture using feeder cells was carried out to maintain differentiation pluripotency similarly to the conventional method. The figure shows results of flow cytometry performed for the second passage.

The lower-left histogram (P2) was obtained from cells on the tenth day according to this Example. The passage was the second passage. The lower-right histogram (P4) was obtained from cells on the 20th day according to this Example. The passage was the fourth passage.

In the histogram, plotted one dot (hereinafter referred to as a plot) represents one cell. A population part of red plots (a light-colored part) located in the upper-left area (P4) in the histogram indicates a population of cells maintaining the function of iPS cells. Other black plot parts (dark-colored parts) indicate cells in which expression levels of iPS cell markers are low.

The upper-right histogram (NC) shows a result of negative control based on cells that are not iPS cells. It is a distribution (black plots) deviated from the area (P4) having the function of iPS cells.

As shown in the lower part of FIG. 13, most of iPS cells obtained by culturing them on a plate having compartments exhibited TRA-1-60 positive even on the 10th day (the 2nd passage) and the 20th day (the 3rd passage). The strength of TRA-1-60 positive cells or the ratio of these cells to all the cells was comparable to that in the positive control.

The result of the FACS test shows that even when a plurality of times of passage are required to prepare a population of cell aggregates of pluripotent stem cells, the method according to this Example can maintain the high uniformity of undifferentiated states among the cell aggregates. Even when feeder cells were not used, the undifferentiated states of iPS cells were maintained by using a plate having compartments.

### <Antibody Staining>

Cell aggregates of iPS cells obtained on the tenth day in the culturing were recovered by the above-described contact method and collected in a 15 ml tube. After the cells were individualized by treating the cell aggregates by TrypLE Select in a manner similar to the above-described process, the tube was centrifuged at 270 G and then a supernatant was removed. After iPS cells were suspended in an appropriate medium, the iPS cells were sown on feeder cells that had been cultured on a 6-well plate in advance.

Five to seven days after the cell seeding, the iPS cells on the feeder were stained according to the following procedure.
1. The medium was removed from each well of the 6-well plate and 1 ml of PBS was added in each well.
2. The PBS was removed and 500 μl of 4% PFA (paraformaldehyde) was added.
3. The cells were reacted with PFA in a refrigerator at 4° C. for 15 minutes.
4. The PFA was removed from the well and 1 ml of PBS was added.
5. A primary antibody was diluted with PBS containing a 5% CCS (Cosmic Calf Serum) and 0.1% Triton by a factor of 200. Then, 500 μl of this diluted antibody solution was added in the well. The primary antibody was composed of an anti-OCT3/4 antibody (C-10, SC-5279, Santacruz) and an anti-NANOG antibody (abcam, ab2162).
6. The antibody and the cells were reacted at a room temperature for one hour.
7. The diluted antibody solution was removed and the well was washed with 1 ml of PBS. The well was washed with PBS again.
8. A secondary antibody was diluted with PBS containing a 5% CCS (Cosmic Calf Serum) and 0.1% Triton by a factor of 1,000 and this diluted antibody solution was added in the well. The secondary antibody was composed of Donkey anti-Mouse IgG (H+L) Secondary Antibody, Alexa Fluor 488 conjugate, and Donkey anti-goat IgG (H+L) Secondary Antibody, Alexa Fluor 647 conjugate. Alexa Fluor is a trademark.
9. The antibody and the cells were reacted at a room temperature for 30 minutes.
10. The well was washed twice with PBS. Cells were observed by using a fluorescence microscope EVOS (Thermo Fisher Scientific).

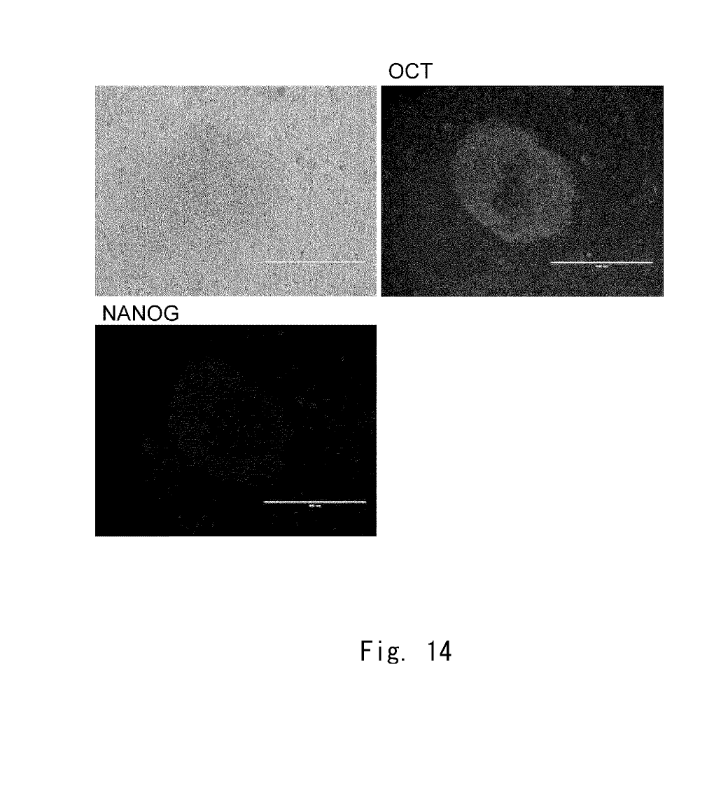

FIG. 14 shows observation images of cells by using antibody staining. The length of bars in the cross direction shown in the observation images represents 400 μm. The upper-left image is an image of a bright field. The upper-right image is a result of staining for Oct3/4. The lower-left image is a result of staining for Nanog.

Based on the result of the staining, it was found that in the iPS cells cultured on the plate having compartments, OCT3/4 and NANOG, which are marker genes of pluripotent stem cells, were expressed (OCT3/4 and NANOG were positive). From this result, it was indicated that the iPS cells cultured on the plate having compartments maintained differentiation pluripotency.

<Comparison with Planar Culturing>

In this test, the above-described iPS cell lines, i.e., Line 1, Line 2, and Line 3 were used.

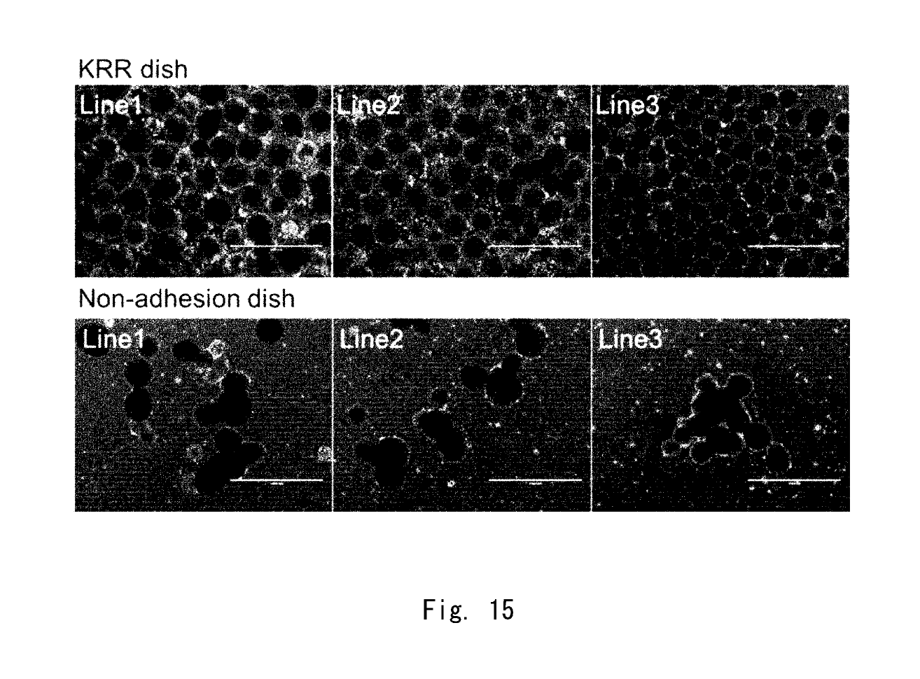

FIG. 15 shows observation images of recovered populations of cell aggregates. The observation images (KRR Dish) on the upper part show results of subculture of respective cells on plates having compartments. The observation images (Non-adhesion dish) on the lower part show results of subcultures of respective cells on planar dishes that had been subjected to a cell low-adhesion treatment. Feeder cells were not used in any of the subcultures. Further, the number of times of passage was one (P1) in each of the subcultures. As shown in FIG. 15, it was found that the method according to this example contributed to making the sizes of cell aggregates uniform in the preparation of populations of cell aggregates of pluripotent stem cells.

In general, iPS cells have a tendency to be differentiated when their sizes reach a certain size or larger. Further, nutrition in a medium is less likely to diffuse into inside of cell aggregates. Therefore, sizes of cell aggregates composed of cells cultured on a planar dish subjected to a cell low-adhesion treatment were nonuniform. Further, in these cells, differentiation and induction of cell deaths were induced. In contrast to this, cells cultured on a plate having compartments did not suffer such adverse effects. It is considered that the culture method according to this example is more suitable for culturing of iPS cells than the related-art planar culturing method.

### Example 6

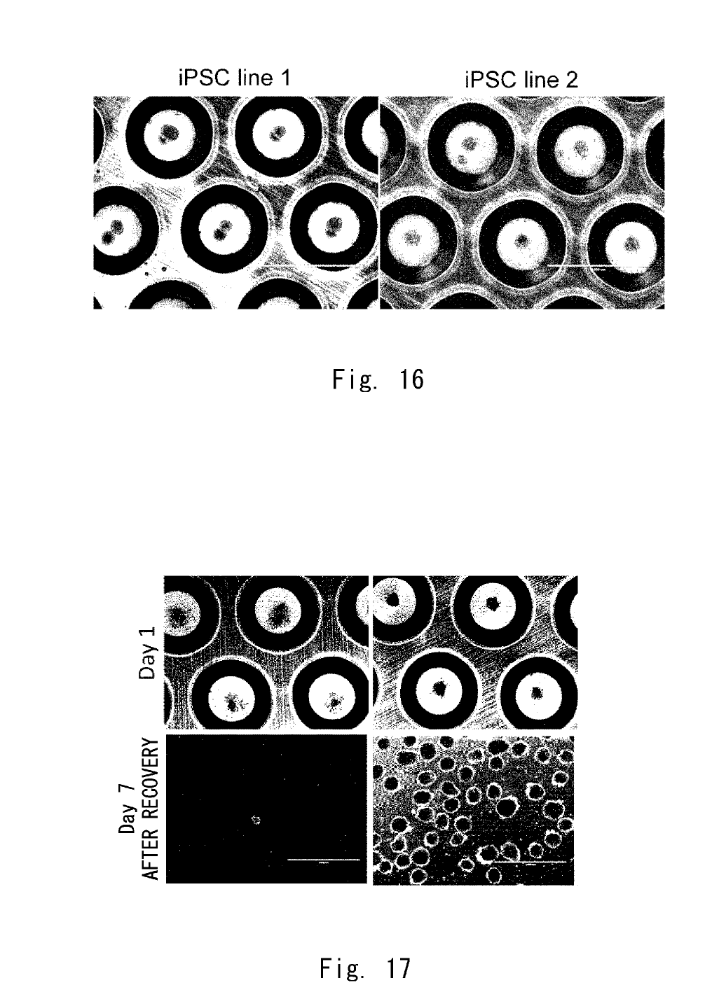

In this Example, the above-described iPS cells Line 1 and Line 2 were cultured in a manner similar to that of Example 4. FIG. 16 shows an observation image of cell aggregates of iPSC Line 1 on the left side. Further, FIG. 16 shows an observation image of cell aggregates of iPSC Line 2 on the right side. The passage was the first passage. It was the fifth day after the first seeding on the plate. In this Example, similarly to Example 4, it was possible to make the sizes of cell aggregates uniform in the preparation of populations of cell aggregates of pluripotent stem cells. Therefore, it was found that the method according to the above-described example contributed to obtaining cell aggregates having uniform sizes irrespective of the type of cell line.

### Example 7

In this Example, instead of using the Lines 1, 2 and 3, a cell line Line 4 of iPS cells that express un-differentiation markers, like the aforementioned cell lines, and have a tendency to be differentiated into three germ layers was used. Cells were cultured under conditions similar to those in Example 4, except for the above-described matter.

FIG. 17 shows observation images of the Line 4 sown on a plate according to this example. The upper part of FIG. 17 shows the Line 4 immediately after the seeding. The lower part shows the Line 4 when it was recovered from the plate on the seventh day. As shown on the left side of FIG. 17, when the Line 4 was cultured by a method similar to that of Example 1, efficiency with which the Line 4 had formed cell aggregates was lower than those of the Lines 1, 2 and 3. However, the line-by-availability was maintained. The inventors have presumed that the composition of the Medium A was insufficient to clump or assemble cells of Line 4.

Culturing was carried out by using a medium that was obtained by adding extracellular matrixes in the Medium A. As shown in the right side of FIG. 17, the Line 4 had formed cell aggregates.

In this Example, commercially-available Matrigel (Trademark) was added in the Medium A so that its concentration became 10 μL/mL or higher. It is considered that the concentration of the extracellular matrixes in the medium should be in a range equal to or higher than 10 μL/mL. It is considered that the extracellular matrixes may be one of Lamin 551, which are modified types of Matrigel, laminin, collagen, fibronectin, vitronectin, and lamin, or a combination thereof. Other conditions were similar to those in the example.

The above-described result shows that it is possible to clump or assemble even the cells, which do not form cell aggregates in a medium containing no extracellular matrix, by using a medium containing extracellular matrixes added therein.

### Example 8

In this Example, by using the expression strength of TRA-1-60 as a reference, culturing using compartments according to this example was compared with culturing on a planar surface coated with extracellular matrixes in the related art.

### <Preparation of Cell>

### (W/Feeder)

A dish coated with extracellular matrixes was used as a positive control. This positive control is hereinafter referred to as “w/feeder”.
1. Preparation of culture vessel: a Matrigel was used as extracellular matrixes with which a dish should be coated. To obtain a solution, 180 μl of Matrigel was added in 12 ml of DMEM on ice. A proper amount of the solution was poured in each of 12 dishes of a culture plate (hereinafter this culture vessel is simply referred to as a dish). The dish was placed in a CO₂ incubator for one hour or longer. The medium was removed immediately before the dish was used. Hereinafter, this dish is called an extracellular-matrix dish.
2. Through a procedure similar to that in Example 1, iPS cells were cultured by using feeder cells. After that, only the feeder cells were removed from the resulting product of the culturing. Next, iPS cells were cultured in each well of the 6-well plate. After the culturing, 1 ml of a medium Y was poured in each well. The iPS cells were peeled off from the well by using a scraper. Suspending was sufficiently performed until the iPS cells were individualized into single cells.
3. For the extracellular-matrix dish, which had been prepared as described above, 2×10⁵ iPS cells were sown in each dish. The extracellular-matrix dish was incubated in a CO₂ incubator. A medium obtained by adding a ROCK inhibitor in the Medium A in a ratio of 1:1,000 was used.
4. One day after the seeding, the extracellular-matrix dish was removed from the incubator. After that, medium replacement was carried out every day by using the same medium as the above-described medium, i.e., the medium obtained by adding the ROCK inhibitor in the Medium A.
5. After seven to ten days had elapsed from the seeding, the medium was removed from the extracellular-matrix dish. Then, 1 ml of PBS was added in the dish. The PBS was removed by using an aspirator. Then, 500 μl of TrypLE Select was added in the dish. The dish was incubated for five minutes in the CO₂ incubator.
6. After the incubation, 500 μl of the medium Y was added in the dish. By performing suspending by using a Pipetman, iPS cells were individuated into single cells.

### (W/O Feeder)

Planar culturing was performed as a comparative example. A planar dish that had been subjected to a cell low-adhesion treatment was used. Hereinafter, this comparative example is referred to as “w/o feeder”.

The planar dish subjected to the cell low-adhesion treatment was similar to that shown in <Comparison with Planar Culturing> in [Example 5]. The culturing on the planar dish was continued for seven to ten days after the seeding. The number of times of passage was one (P1).

After the culturing, a cell suspension solution was recovered in a 15 ml tube. After centrifuging the tube at 270 G, a supernatant was sucked out and thereby removed. After 500 μl of TrypLE Select was added in the tube, the tube was incubated in an incubator at 37° C. for ten minutes. After the incubation, 500 μl of the medium Y was added in the tube. By suspending the tube and cells by using a Pipetman, iPS cells were individuated into single cells.

### (KRR)

Induced pluripotent stem (iPS) cells were cultured on a plate having compartments according to this example. Hereinafter, this example is referred to as “KRR”.

Through a procedure similar to that in Example 4, iPS cells were cultured on the plate having compartments according to this example for seven to ten days after the seeding. Through a procedure similar to that in Example 5, iPS cells were recovered in a 15 ml tube. After centrifuging the tube at 270 G, a supernatant was sucked out and thereby removed. After 500 μl of TrypLE Select was added, the tube was incubated in an incubator at 37° C. for ten minutes. After the incubation, 500 μl of the medium Y was added in the tube. By suspending the tube and cells by using a Pipetman, iPS cells were individuated into single cells.

### <FACS>

After cells according to the above-described w/feeder, the w/o feeder, and the KRR were prepared, they were analyzed through the following procedure.
1. The iPS cells, which had been individualized and became single cells, were recovered in a 1.5 ml tube. The number of cells was counted by using a hemocytometer. After that, the tube was centrifuged at 270 G and then a supernatant was removed.
2. For 5×10⁵ iPS cells, 50 μl of PBS was added. In the PBS, 2.5 μl of an anti-TRA-1-60 antibody had been added in advance. The anti-TRA-1-60 antibody had been chemically treated in advance so as to generate fluorescence. The tube was incubated at a room temperature for 30 minutes under a light-shielded condition.
3. After the incubation, 1 ml of PBS was added in the tube. After centrifuging the tube at 270 G, a supernatant was removed from the tube.
4. Fluorescence intensities of TRA-1-60-positive cells were analyzed by using a flow cytometer CytoFlex.

### <Result>

It was shown that expression of un-differentiation markers of the iPS cells cultured on the plate having compartments could be maintained at a higher level than that of the iPS cells cultured on the extracellular-matrix dish as shown below.

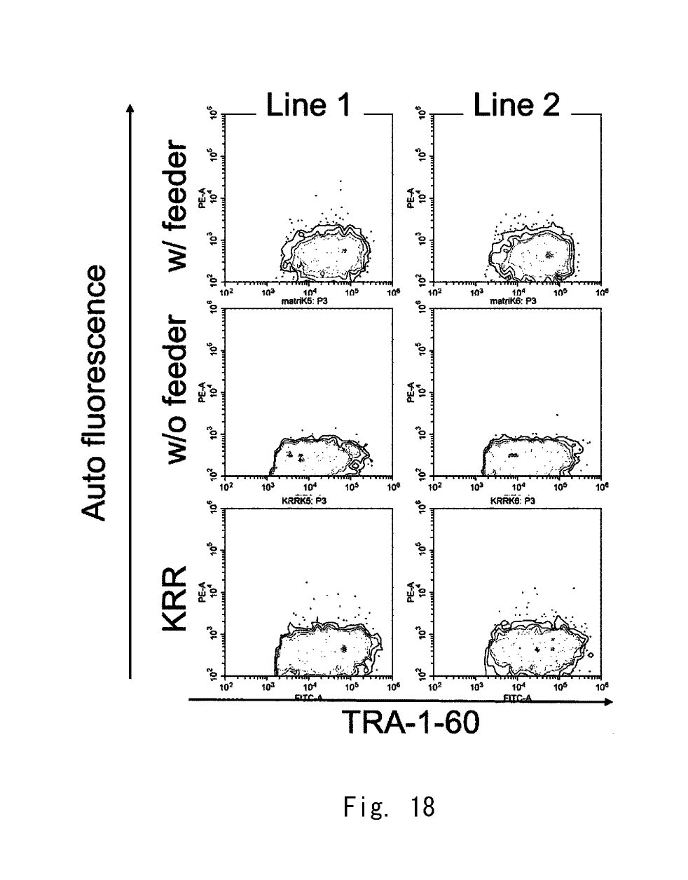

FIG. 18 shows 2-parameter histograms of ACS. The vertical axis indicates the intensity of Auto-fluorescence. The horizontal axis indicates the fluorescence intensity of TRA-1-60. In the KRR, patterns similar to those of the W/feeder were obtained.

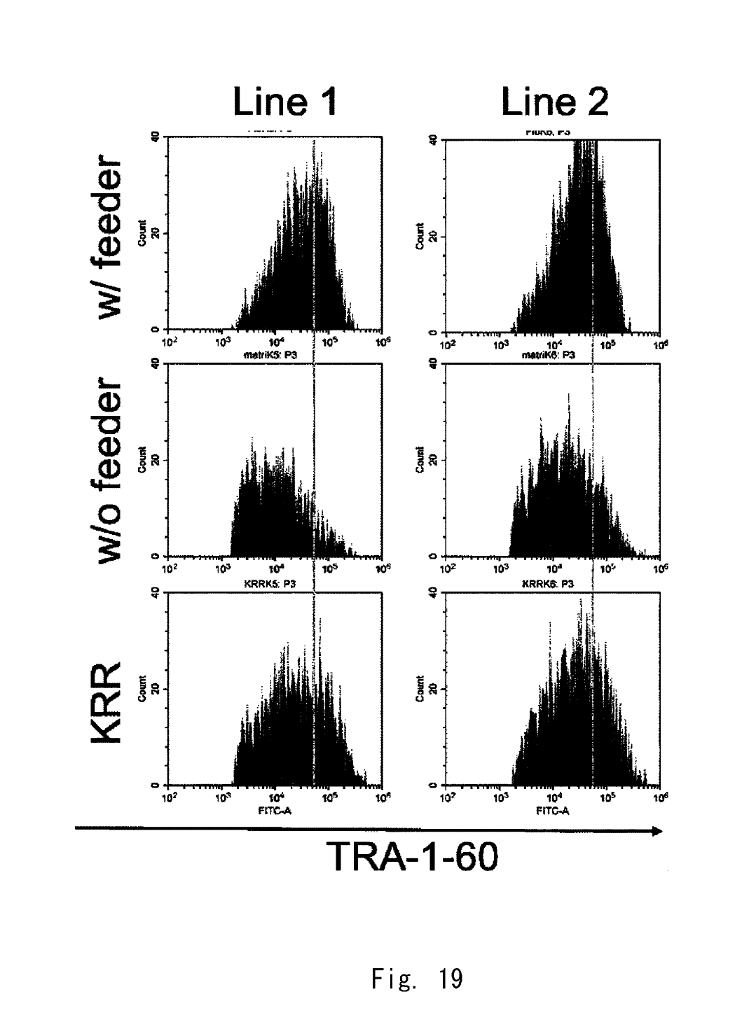

FIG. 19 shows 1-parameter histograms of FACS. The horizontal axis indicates the fluorescence intensity of TRA-1-60. The “count” on the vertical axis represents the number of cells. In the KRR, patterns similar to those of the W/feeder were obtained.

Based on the results shown in FIGS. 18 and 19, it was found that the KRR exhibited histograms comparable to those of the w/feeder. For example, it can be determined based on positions of darkest gray parts in the histograms.

### Example 9

Expression rates of un-differentiation markers of each cluster of cells cultured on the plate having compartments were measured.

Through a procedure similar to that in Example 4, cells were cultured for seven to ten days after the seeding (P1). When cell aggregates were recovered from the plate, they were recovered one by one. Hereinafter, each of these cell aggregates is referred to as a single clump or a clump. Then, 10 to 12 single clamps were recovered in a 1.5 ml tube in which 300 μl of TrypLE Select was poured in advance.

The tube was incubated at 37° for 10 minutes. After the incubation, 700 μl of PBS was added in the tube. Cells were suspended 10 to 30 times. The tube was centrifuged at 270 G. After that, it was processed according to the procedures 8. to 10. in <Antibody Staining> of [Example 5].

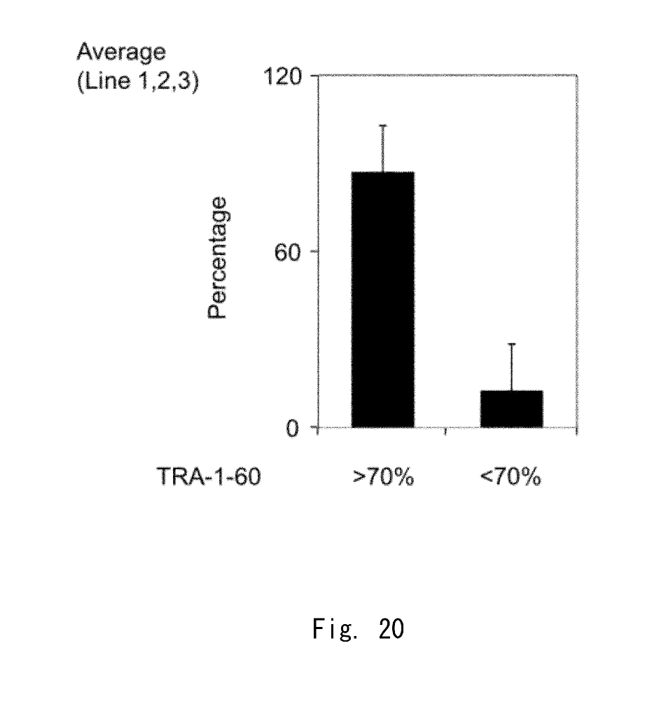

FIG. 20 is a graph showing expression intensities of TRA-1-60, showing results of analyses on obtained stained images. TRA-1-60 positive rates were 70% or higher in 80% or more of 10 to 12 clumps.

### Example 10

Differentiation pluripotency of each clamp cultured on the plate having compartments was tested.
1. By a method similar to that in Example 4, iPS cells were cultured for three days. The medium was replaced by a medium Y containing no bFGF. Then, iPS cells were further cultured for seven days. The medium was replaced once every two days.
2. After the above-described seven-day culturing was completed, clusters of iPS cells were recovered from the plate having compartments. The iPS cells were sown in a 10 cm dish coated with gelatin. After that, the iPS cells were further cultured for seven days. The medium was replaced once every two days.
3. After the above-described seven-day culturing was completed, immunostaining was carried out according to the below-shown protocol by using the below-shown antibodies.
4. After washing the dish with PBS, the PBS was removed and 500 μl of PBS containing 4% PFA was added in the dish.
5. The PFA and cells were reacted in a refrigerator at 4° C. for 15 minutes.
6. The PFA was removed from the dish and 1 ml of PBS was added
7. A primary antibody was diluted by PBS containing 5% CCS and 0.1% Triton. Then, 500 μl of the diluted antibody solution was added in the dish. As the primary antibody, an antibody that was obtained by diluting a TUJI-1 antibody, a FOXA2 monoclonal antibody, and a Brachyury antibody by a factor of 200 was used.
8. The antibody and cells were reacted at a room temperature for one hour.
9. The diluted antibody solution was removed from the dish. Cells were washed with 1 ml of PBS. Washing was carried out again.
10. A secondary antibody was diluted by PBS containing 5% CCS and 0.1% Triton by a factor of 1,000. The following antibodies were used as the secondary antibody.

Donkey anti-rat IgG (H+L) Secondary Antibody, Alexa Fluor 488 conjugate

Donkey anti-mouse IgG (H+L) Secondary Antibody, Alexa Fluor 555 conjugate

Donkey anti-goat IgG (H+L) Secondary Antibody, Alexa Fluor 647 conjugate
11. The diluted secondary antibody solution and cells were reacted at a room temperature for 30 minutes.
12. Cells were washed twice with PBS. Cells were observed by using a fluorescence microscope EVOS.

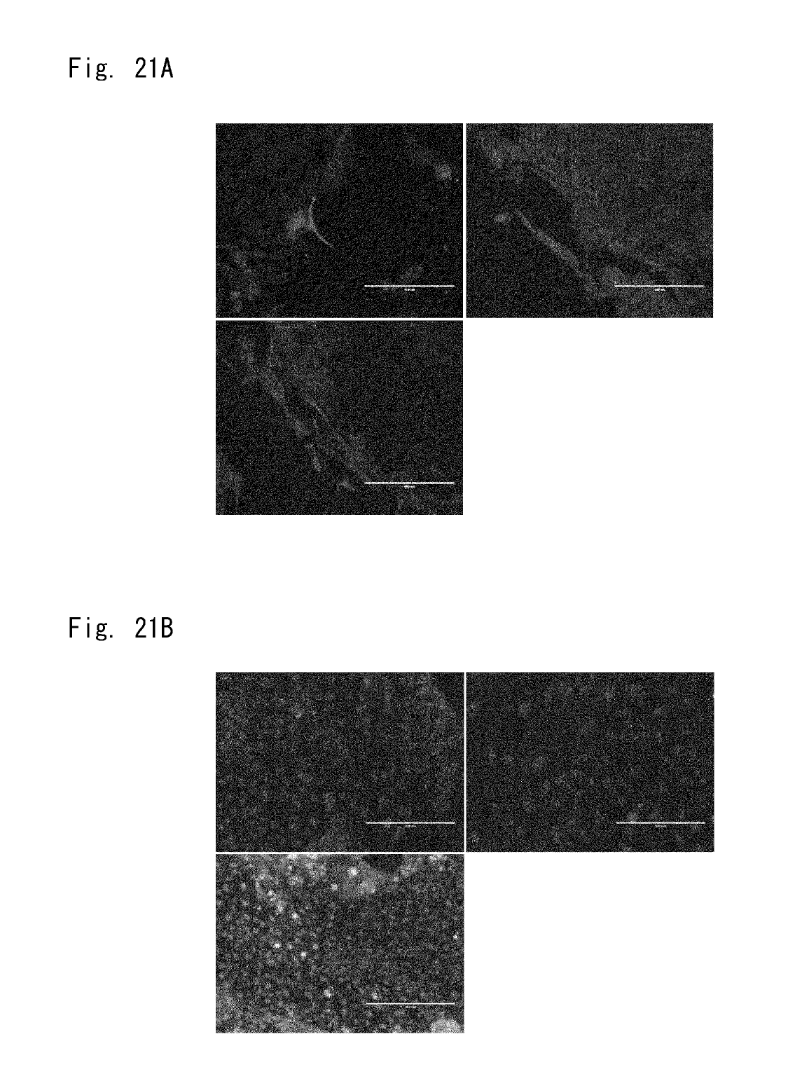

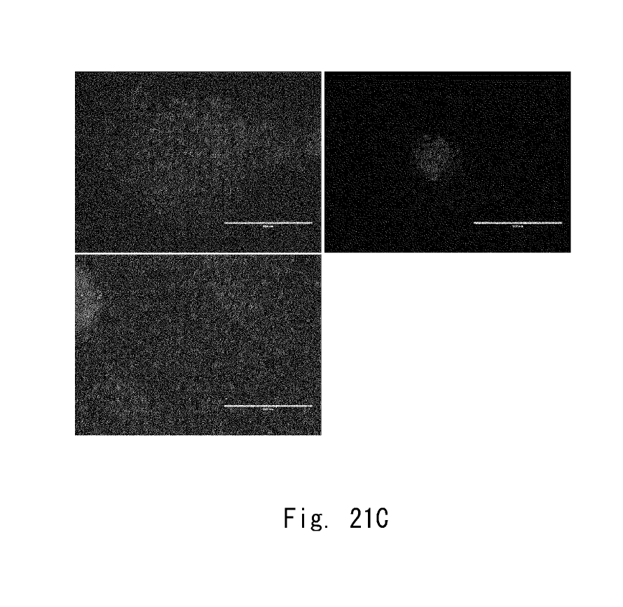

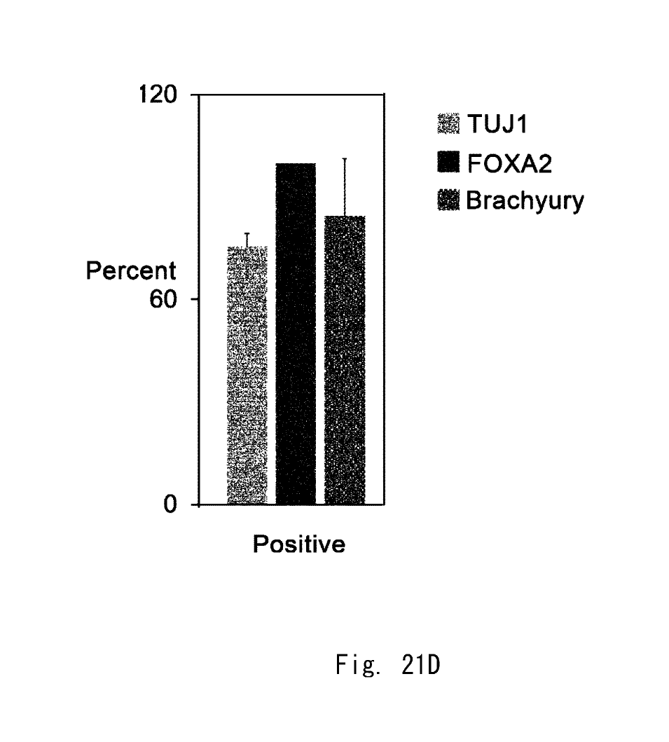

FIGS. 21A to 21C show fluorescence observation images of cell aggregates.

FIGS. 21A, 21B, and 21C show staining patterns of TUJI-1, FOXA2, and Brachyury, respectively. In each of FIGS. 21A to 21C, the upper-left, upper-right, and lower-left images correspond to the Line 1, the Line 2, and the Line 3, respectively. TUJ-1 is a differentiation marker for ectoderms. The results shown in FIG. 21A indicate that cells in cell aggregates have a capability of inducing differentiations into cells that are generated from ectoderms such as nerve cells. FOXA1 is a differentiation marker for endoderms. FOXA1 is a differentiation marker necessary, in particular, in the earliest process of hepatic tissue formation. The results shown in FIG. 21B indicate that cells in cell aggregates have a capability of inducing differentiations into cells generated from endoderms such as a liver. Brachyury is a differentiation marker for early mesoderms. The results shown in FIG. 21B indicate that cells in cell aggregates have a capability of inducing differentiations into cells generated from mesoderms such as a muscle.

FIG. 21D is a bar graph showing ratios of the numbers of cells in which respective embryoid markers are expressed based on the total number of cells. The graph indicates that a ratio of embryoid bodies induced from cell aggregates by the in-vitro differentiation inducting system is 80% or higher.

[24] The method for preparing a population of cell aggregates of stem cells according to the above item [2], further including:

mixing the cell aggregates formed by clumping or assembling clusters of cells again without breaking up the cell aggregates;

distributing the mixed cell aggregates into each of two or more compartments;

bringing the two or more mixed cell aggregates close to each other in each of the compartments; and

further clumping or assembling the two or more cell aggregates, the two or more cell aggregates having been brought close to each other.

[25] The method for preparing a population of cell aggregates of stem cells according to the above item [2], further including:

mixing the two or more formed cell aggregates with each other after the cell aggregates are formed and before the cell aggregates are broken up;

distributing the mixed cell aggregates into each of two or more compartments;

bringing the two or more mixed cell aggregates close to each other in each of the compartments;

further clumping or assembling the two or more cell aggregates, the two or more cell aggregates having been brought close to each other; and

repeating a process of forming cell aggregates larger than the mixed cell aggregates once or twice or more.

[26] The method for preparing a population of cell aggregates of stem cells according to the above item [1], further including:

mixing the two or more formed cell aggregates with each other;

distributing the mixed cell aggregates into each of two or more compartments;

bringing the two or more mixed cell aggregates close to each other in each of the compartments; and

further clumping or assembling the two or more cell aggregates, the two or more cell aggregates having been brought close to each other.

[27] The population of cell aggregates according to the above item [19], in which when in-vivo induced differentiation is performed, a ratio of cell aggregates forming teratoma differentiated into three germ layers is 80% or higher.

[28] The population of cell aggregates according to the above item [19], in which

when ten cell aggregates are selected from the population and the cell aggregates are induced to be differentiated into endoderms by an in vitro differentiation-inducing system, and

when it is determined as to whether or not at least one of endoderm markers FOXA2 and AFP is positive for the cell aggregates,

a positive rate of the endoderm marker is 80% or higher.

[29] The population of cell aggregates according to the above item [19], in which when ten cell aggregates are selected from the population and the cell aggregates are induced to be differentiated into mesoderms by an in vitro differentiation-inducing system, and

when it is determined as to whether or not at least one of mesoderm markers Brachyury and MSX1 is positive for the cell aggregates,

a positive rate of the mesoderm marker is 80% or higher.

[30] The population of cell aggregates according to the above item [19], in which

when ten cell aggregates are selected from the population and the cell aggregates are induced to be differentiated into ectoderms by an in vitro differentiation-inducing system, and

when it is determined as to whether or not at least one of ectoderm markers Pax6, SOX2, PsANCAM and TUJ1 is positive for the cell aggregates by measuring a gene expression level of each individual embryoid body by a PCR method,

a positive rate of the ectoderm marker is 80% or higher.

[31] A method for preparing a population of cell aggregates of stem cells including:

distributing two or more pre-aggregation units into each of two or more compartments having a uniform size, the pre-aggregation units being at least either clusters of cells or single cells;

bringing the two or more pre-aggregation units close to each other in each of the compartments; and

allowing the two or more pre-aggregation units brought close to each other to be clumped or assembled and grow to form a cell aggregate, in which

the pre-aggregation units to be distributed are separated from and mixed with each other, and each of the clusters of cells is formed of stem cells.

[32] The method for preparing a population of cell aggregates of stem cells according to the above item [1] or [31], in which the stem cells are pluripotent stem cells.

[33] The method for preparing a population of cell aggregates of stem cells according to the above item [31] or [32], in which the pre-aggregation units are clusters of cells.

This application is based upon and claims the benefit of priority from U.S. Provisional Application No. 62/272,524, filed on Dec. 29, 2015, the disclosure of which is incorporated herein in its entirety by reference.

### REFERENCE SIGNS LIST

20 CULTURING DEVICE; 21-26 STEPS; 27 ARROW; 29 PARTITION WALL; 30 PLATE; 31A, 31B HOLES; 32A, 32B PARTITIONS; 33A, 33B TOP OPENINGS; 34A, 34B BOTTOM OPENINGS; 35 CULTURE SOLUTION; 36A, 36B DROPLETS; 37 RESERVOIR COMPARTMENT; 38 SUSPENSION SOLUTION; 39 THRESHOLD; 40 CELL AGGREGATE; 41 POPULATION; 42A-42C CLUSTERS OF CELLS; 43A-43C CELL AGGREGATES; 44A, 44B POPULATIONS; 45 SUPPORT; 46 SIDE WALL; 47 FLANGE; 50 CHAMBER; 55 TRAY; 56 SIDE WALL; 57 BOTTOM; 58 SPACE; 60 TRAY; 65 RECOVERY SOLUTION

1. A method for preparing a population of cell aggregates of stem cells, comprising:
distributing two or more clusters of cells into each of two or more compartments having a uniform size;
bringing the two or more clusters of cells close to each other in each of the compartments; and
allowing the two or more clusters of cells brought close to each other to be clumped or assembled and grow to form a cell aggregate, wherein
the clusters of cells to be distributed are separated from and mixed with each other, and each of the clusters of cells is formed of stem cells.

2. The method for preparing a population of cell aggregates of stem cells according to claim 1, further comprising:
generating clusters of cells by breaking up the formed cell aggregates;
mixing the clusters of cells generated from the different cell aggregates;
distributing two or more mixed clusters of cells into each of two or more compartments;
bringing the two or more mixed clusters of cells close to each other in each of the compartments; and
clumping or assembling the two or more clusters of cells again, the two or more clusters of cells having been brought close to each other.

3. The method for preparing a population of cell aggregates of stem cells according to claim 2, wherein the cell aggregates are broken up when each of the cell aggregates has a diameter equal to or smaller than 1 mm.

4. The method for preparing a population of cell aggregates of stem cells according to claim 2, wherein during the growth of the cell aggregates, the cell aggregates are allowed to grow for a period of from 2 to 14 days.

5. The method for preparing a population of cell aggregates of stem cells according to claim 2, wherein during the growth of the cell aggregates, the cell aggregates are allowed to grow for a period of from 3 to 7 days.

6. The method for preparing a population of cell aggregates of stem cells according to claim 2, wherein a process of breaking up the cell aggregates, mixing the clusters of cells, bringing the clusters of cells close to each other, and clumping or assembling the clusters of cells again is repeated once or twice or more.

7. The method for preparing a population of cell aggregates of stem cells according to claim 1, wherein
the stem cells are cultured in a plate to form a colony,
the clusters of cells are generated by dissociating the colony,
the generated clusters of cells are mixed, and
the clusters of cells are used for the distribution.

8. The method for preparing a population of cell aggregates of stem cells according to claim 7, wherein
the colony is broken up by physical dissociation, and
an enzyme treatment is not applied onto the colony.

9. The method for preparing a population of cell aggregates of stem cells according to claim 7, wherein
the colony is broken up only by an enzyme treatment, and
physical dissociation is not applied onto the colony.

10. The method for preparing a population of cell aggregates of stem cells according to claim 7, wherein when the colony is broken up, an enzyme treatment and physical dissociation are applied onto the colony.

11. The method for preparing a population of cell aggregates of stem cells according to claim 1, wherein
the compartments are each formed by a hole of a plate,
the hole is one of a through-hole and a recess,
the hole has a top opening formed in a top face of the plate,
the top openings of the holes of the compartments have an equal area, and
the top opening has a diameter of 1.5 mm or less.

12. The method for preparing a population of cell aggregates of stem cells according to claim 1, wherein
the compartments are each formed by a through-hole of a plate,
the through-hole has a bottom opening formed in a bottom of the plate,
the bottom opening has a diameter of 1 mm or less, and
the cell aggregates are recovered from the plate by causing the cell aggregates to pass through the bottom opening.

13. The method for preparing a population of cell aggregates of stem cells according to claim 12, wherein
the clusters of cells are cultured in a culture solution dispensed in the compartment;
the culture solution forms a droplet;
the droplet adheres to the bottom opening and projects from the bottom opening so as to hang down therefrom; and
the bottom of the compartment is formed by a meniscus of the droplet.

14. The method for preparing a population of cell aggregates of stem cells according to claim 1, wherein
an inscribed sphere in each of the compartments has a diameter in a range from 5×10¹ μm to 1×10³ μm, and
the inscribed sphere contacts the bottom of the corresponding compartment.

15. The method for preparing a population of cell aggregates of stem cells according to claim 1, wherein
the clusters of cells are cultured in a culture solution dispensed in each of the compartments,
the culture solution is joined with a culture solution dispensed in a reservoir compartment through a top portion of each of the compartments, and
no cells are present in the culture solution in the reservoir compartment.

16. The method for preparing a population of cell aggregates of stem cells according to claim 1, wherein
the compartments are each formed by a hole of a plate,
the hole is one of a through-hole and a recess,
the hole has a top opening formed in a top face of the plate, and
during the distribution, the top face is covered with a suspension containing the clusters of cells.

17. The method for preparing a population of cell aggregates of stem cells according to claim 16, wherein the suspension contains one to 5000 clusters of cells per unit area (1 cm²) of the top face.

18. The method for preparing a population of cell aggregates of stem cells according to claim 1, wherein
the clusters of cells are cultured in a culture solution dispensed in the compartment, and
extracellular matrixes are suspended or dissolved in the culture solution.

19. A cell culture method comprising:
forming a cell aggregate from stem cells; and
differentiating the stem cells while performing suspension culturing of adherent culturing, wherein,
during the formation of the cell aggregate, two or more clusters of cells are distributed into each of two or more compartments having an equal size,
the two or more clusters of cells are brought close to each other in each of the compartments,
the two or more clusters of cells brought close to each other are clumped or assembled and allowed to grow to form a cell aggregate,
before the distribution, the clusters of cells are separated from each other and are mixed with each other, and
each of the clusters of cells includes stem cells.

20. The cell culture method according to claim 19, wherein cells in the cell aggregates are further differentiated into one of ectoderms, mesoderms, and endoderms in the compartment.

21. A population of cell aggregates, wherein:
10 cell aggregates are selected from the population,
10 or more cells are selected from cells in the selected cell aggregates,
a positive rate of the 10 or more cells is measured by determining whether or not at least one of pluripotent stem cell markers of Nanog, Oct3/4, and TRA-1-60 is positive for the cells, and
when the positive rate is measured from the population three times, an average of the three positive rates is 80% or higher.

22. The population of cell aggregates according to claim 21, wherein ten cell aggregates are selected from the population,
when it is determined as to whether or not at least one of pluripotent stem cell markers of Nanog, Oct3/4, and TRA-1-60 is positive for the ten selected cell aggregates, a positive rate of the marker is 80% or higher.

23. The population of cell aggregates according to claim 21, wherein a ratio of embryoid bodies induced from the cell aggregates by an in vitro differentiation-inducing system is 80% or higher, and the embryoid bodies are cell aggregates containing mixed tissues of three germ layers.
